(19) United States
(12) Patent Application Publication
(10) Pub. No.: US 2021/0388028 A1
(43) Pub. Date: Dec. 16, 2021

(54) GLYCOPEPTIDE DERIVATIVE COMPOUNDS AND USES THEREOF

(71) Applicant: INSMED INCORPORATED, Bridgewater, NJ (US)

(72) Inventors: Donna Konicek, Bridgewater, NJ (US); Adam Plaunt, Bridgewater, NJ (US); Vladimir Malinin, Bridgewater, NJ (US); Walter Perkins, Bridgewater, NJ (US); Ryan Heckler, Bridgewater, NJ (US)

(21) Appl. No.: 17/295,370

(22) PCT Filed: Nov. 20, 2019
(86) PCT No.: PCT/US2019/062317

§ 371 Date: May 19, 2021

Related U.S. Application Data
(60) Provisional application No. 62/770,489, filed on Nov. 21, 2018.

(51) Int. Cl. C07K 9/00

(52) U.S. Cl. CPC C07K 9/008; A61K 38/00

(57) ABSTRACT

Provided herein are compounds, compositions and methods for the treatment of Gram positive bacterial infections. The infection in some embodiments, is a pulmonary infection. The method for treating the bacterial infection, comprises in one embodiment, administering to a patient in need thereof, a composition comprising an effective amount of a compound a glycopeptide derivative of Formula (I) or (II), or a pharmaceutically acceptable salt of Formula (I) or (II). The bacterial infection can comprise intracellular bacteria, planktonic bacteria and/or bacteria present in a biofilm.

### BACKGROUND OF THE INVENTION

The high frequency of multidrug resistant bacteria, and in particular, Gram-positive bacteria, both in the hospital setting and the community present a significant challenge for the management of infections (Krause et al. (2008). Antimicrobial Agents and Chemotherapy 52(7), pp. 2647-2652, incorporated by reference herein in its entirety for all purposes).

The treatment of invasive Staphylococcus aureus (S. aureus) infections has relied significantly on vancomycin. However, the treatment and management of such infections is a therapeutic challenge because certain S. aureus isolates, and in particular, methicillin-resistant S. aureus isolates, have been shown to be resistant to vancomycin (Shaw et al. (2005). Antimicrobial Agents and Chemotherapy 49(1), pp. 195-201; Mendes et al. (2015). Antimicrobial Agents and Chemotherapy 59(3), pp. 1811-1814, each of which is incorporated by reference herein in its entirety for all purposes).

Because of the resistance displayed by many Gram-positive organisms to antibiotics, and the general lack of susceptibility to existing antibiotics, there is a need for new therapeutic strategies to combat infections due to these bacteria. The present invention addresses this and other needs.

### SUMMARY OF THE INVENTION

In one aspect of the invention, a compound of Formula (I), or a pharmaceutically acceptable salt thereof, is provided:

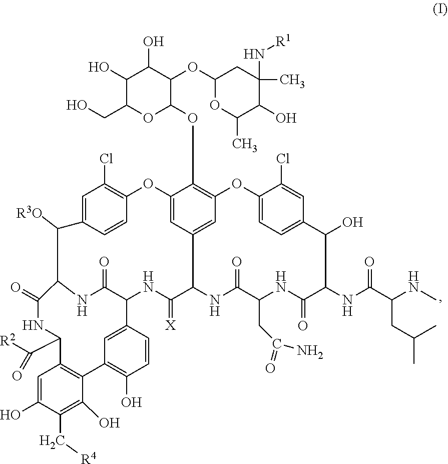

wherein,

R¹ is C₁-C₁₈ linear alkyl, C₁-C₁₈ branched alkyl, R⁵—Y—R⁶—(Z)ₙ, or;

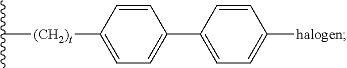

R² is —OH or —NH—(CH₂)_{q}—R⁷;

R³ is H or

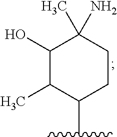 [SMILES: *C1CCC(C)(N)C(O)C1C]

R⁴ is diethanolamine, a monosaccharide, disaccharide, amino acid, or peptide, wherein the peptide has from 2 to 5 amino acids;

n is 1 or 2;

q is 1, 2, 3, 4, or 5;

t is 1, 2, 3, 4, or 5;

X is O, S, NH or H₂;

each Z is, independently, hydrogen, aryl, cycloalkyl, cycloalkenyl, heteroaryl or heterocycl;

R⁵ and R⁶ are independently selected from the group consisting of alkylene, alkenylene and alkynylene, wherein the alkylene, alkenylene and alkynylene groups are optionally substituted with from 1 to 3 substituents selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl

R⁷ is —N(CH₂)₂; —N⁺(CH₂)₃; or

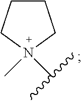

Y is oxygen, sulfur, —S—S—, —NR⁸—, —S(O)—, —SO₂—, —NR⁸C(O)—, —OSO₂—, —OC(O)—, —NR⁸SO₂—, —C(O)NR⁸—, —C(O)O—, —SO₂NR⁸—, —SO₂O—, —P(O)(OR⁸)O—, —P(O)(OR⁸)NR⁸—, —OP(O)(OR⁸)O—, —OP(O)(OR⁸)NR⁸—, —OC(O)O—, —NR⁸C(O)O—, —NR⁸C(O)NR⁸—, —OC(O)NR⁸— or —NR⁸SO₂NR⁸—; and

each R⁸ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, heteroaryl and heterocyclic.

In another aspect, a compound of Formula (II), or a pharmaceutically acceptable salt thereof is provided:

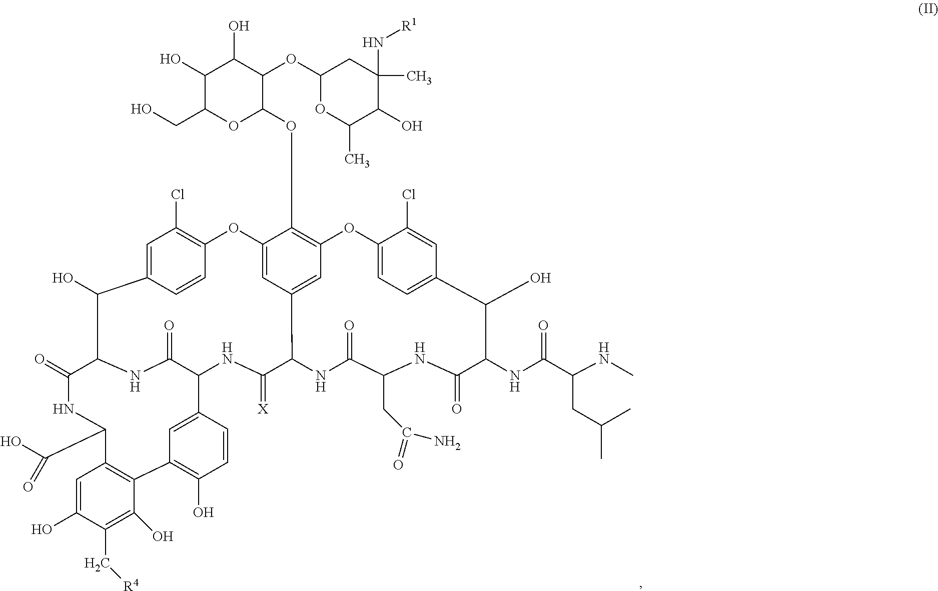

wherein,

R¹ is C₁-C₁₈ linear alkyl, C₁-C₁₈ branched alkyl, R⁵—Y—R⁶—(Z)ₙ, or

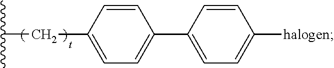

R⁴ is diethanolamine, a monosaccharide, disaccharide, amino acid, or peptide, wherein the peptide has from 2 to 5 amino acids;

n is 1 or 2;

t is 1, 2, 3, 4 or 5;

X is O, S, NH or H₂;

each Z is, independently, hydrogen, aryl, cycloalkyl, cycloalkenyl, heteroaryl or heterocycl;

R⁵ and R⁶ are independently selected from the group consisting of alkylene, alkenylene and alkynylene, wherein the alkylene, alkenylene and alkynylene groups are optionally substituted with from 1 to 3 substituents selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl;

Y is oxygen, sulfur, —S—S—, —NR⁸—, —S(O)—, —SO₂—, —OSO₂—, —NR⁸SO₂—, —SO₂NR⁸—, —SO₂O—, —P(O)(OR⁸)O—, —P(O)(OR⁸)NR⁸—, —OP(O)(OR⁸)O—, —OP(O)(OR⁸)NR⁸—, —NR⁸C(O)NR⁸—, or —NR⁸SO₂NR⁸—; and

each R⁸ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, heteroaryl and heterocyclic.

In one embodiment, a compound of Formula (I), Formula (II), or a pharmaceutically acceptable salt of Formula (I) or Formula (II) is provided, wherein R¹ is C₆ to C₁₆ linear alkyl. In a further embodiment, R¹ is C₆, C₁₀ or C₁₆ alkyl. In even a further embodiment, R¹ is C₁₀ alkyl. In a further embodiment, the bacterial infection is a pulmonary bacterial infection. In even a further embodiment, the administering comprises administering via inhalation.

In one embodiment, a compound of Formula (I), Formula (II), or a pharmaceutically acceptable salt of Formula (I) or Formula (II) is provided, where R¹ is R⁵—Y—R⁶—(Z)ₙ and R⁴ is an amino acid or diethanolamine. In a further embodiment, R⁵ is —(CH₂)₂—, R⁶ is —(CH₂)₁₀—, X is O; Y is NH, Z is hydrogen and n is 1. As such, one embodiment of the invention includes a compound of Formula (I), Formula (II) or a pharmaceutically acceptable salt thereof, where R¹ is —(CH₂)₂—NH—(CH₂)₉—CH₃ and R⁴ is an amino acid or diethanolamine. In a further embodiment, R⁴ is an amino acid selected from D-alanine, β-alanine, aspartic acid, glutamic acid, glycine and iminodiacetic acid. In one embodiment, a patient is treated for a bacterial infection with one of the aforementioned compounds. The bacterial infection is a pulmonary bacterial infection in one embodiment. In even a further embodiment, the administering comprises administering via inhalation.

In one embodiment, a compound of Formula (I), Formula (II), or a pharmaceutically acceptable salt of Formula (I) is provided where R¹ is —(CH₂)₂—NH—(CH₂)₉—CH₃, R³ is H and R⁴ is an amino acid. In a further embodiment, R² is OH. In a further embodiment, the amino acid is D-alanine, β-alanine, aspartic acid, glutamic acid, glycine and iminodiacetic acid. In one embodiment, a patient is treated for a bacterial infection with one of the aforementioned compounds. In even a further embodiment, the administering comprises administering via the intravenous route or via inhalation. In a further embodiment, X is O.

In one embodiment, a compound of Formula (I), or a pharmaceutically acceptable salt of Formula (I) is provided where R¹ is —(CH₂)₂—NH—(CH₂)₉—CH₃, R² is —NH—(CH₂)_{q}—R⁷, R³ is H and R⁴ is diethanolamine or an amino acid. The amino acid, in one embodiment, is D-alanine, β-alanine, aspartic acid, glutamic acid, glycine or iminodiacetic acid. In a further embodiment, compound is administered to a patient in need of treatment of a bacterial infection. In a further embodiment, the compound is administered via the intravenous or pulmonary route (e.g., via inhalation). In a further embodiment, X is O.

In one embodiment a compound of Formula (I) or Formula (II), or a pharmaceutically acceptable salt is provided, where R¹ is

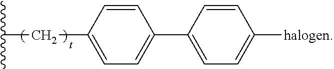

In a further embodiment, R⁴ is diethanolamine or an amino acid. The amino acid, in one embodiment, is D-alanine, β-alanine, aspartic acid, glutamic acid, glycine or iminodiacetic acid. In even a further embodiment, the halogen is Cl and t is 1 or 2. In a further embodiment, X is O and R¹ is

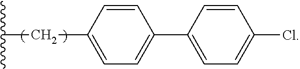

In one embodiment, R⁴ is a monosaccharide. For example, the monosaccharide can be attached to the glycopeptide resorcinol ring via a Mannich reaction. As such, R⁴, in one embodiment, can be selected from one of the following:

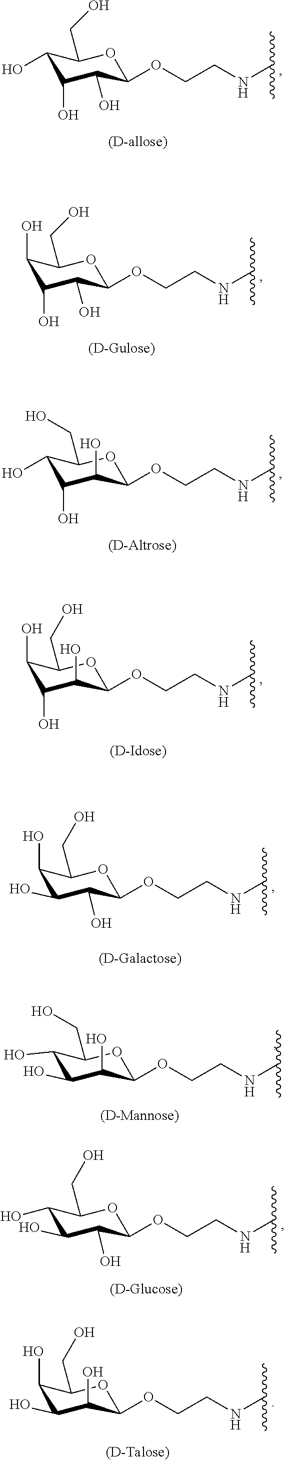

a further embodiment, R¹ is —(CH₂)₂—NH—(CH₂)₉—CH₃.

In one embodiment, R⁴ is

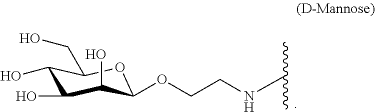

In a further embodiment, R¹ is —(CH₂)₂—NH—(CH₂)₉—CH₃.

In one embodiment of a compound of Formula (I), R¹ is

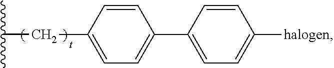

R² is OH and R³ is

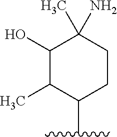 [SMILES: *C1CCC(C)(N)C(O)C1C]

and R⁴ is an amino acid or dipeptide. In even a further embodiment, the halogen is Cl and t is 1 or 2. In a further embodiment, the administering comprises administering via the intravenous route. In a further embodiment, X is O and R¹ is

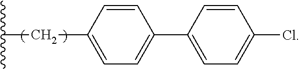

In a further embodiment, R⁴ is an amino acid and is D-alanine, β-alanine, aspartic acid, glutamic acid, glycine or iminodiacetic acid.

In one embodiment of a compound of Formula (I), (II), or a pharmaceutically acceptable salt thereof, R⁴ is an amino acid or peptide. The amino acid, in one embodiment, is D-alanine, β-alanine, aspartic acid, glutamic acid, glycine or iminodiacetic acid.

In one embodiment, R⁴ is diethanolamine. In a further embodiment, X is O and R¹ is —(CH₂)₂—NH—(CH₂)₉—CH₃.

In another aspect of the invention, a method for treating a bacterial infection is provided. The method comprises administering to a patient in need of treatment an effective amount of a compound of Formula (I) or (II), or a pharmaceutically acceptable salt thereof. The bacterial infection can comprise intracellular bacteria, planktonic bacteria and/or bacteria present in a biofilm.

In one embodiment of a method for treating a bacterial infection, the bacterial infection is a Gram-positive cocci infection. In a further embodiment, R¹ is —(CH₂)₂—NH—(CH₂)₉—CH₃. In a further embodiment, the infection is a Gram-positive infection is a cocci infection, and in a further embodiment, is a vancomycin-resistant enterococci (VRE), methicillin-resistant Staphylococcus aureus (MRSA), methicillin-resistant Staphylococcus epidermidis (MRSE), vancomycin resistant Enterococcus faecium also resistant to teicoplanin (VRE Fm Van A), vancomycin resistant Enterococcus faecium sensitive to teicoplanin (VRE Fm Van B), vancomycin resistant Enterococcus faecalis also resistant to teicoplanin (VRE Fs Van A), vancomycin resistant Enterococcus faecalis sensitive to teicoplanin (VRE Fs Van B), or penicillin-resistant Streptococcus pneumoniae (PRSP). In a further embodiment, R⁴ is diethanolamine or an amino acid. The amino acid, in one embodiment, is D-alanine, β-alanine, aspartic acid, glutamic acid, glycine or iminodiacetic acid.

In even another embodiment, a method for treating a bacterial infection with an effective amount of a compound of Formula (I) or (II), or a pharmaceutically acceptable salt thereof is provided. In a further embodiment, the bacterial infection is a Gram-positive cocci infection and R¹ is —(CH₂)₂—NH—(CH₂)₉—CH₃. In a further embodiment, the infection is erythromycin-resistant (erm^{R}), vancomycin-intermediate S. aureus (VISA) heterogenous vancomycin-intermediate S. aureus (hVISA), S. epidermidis coagulase-negative staphylococci (CoNS), penicillin-intermediate S. pneumoniae (PISP), or penicillin-resistant S. pneumoniae (PRSP).

In even another embodiment of the methods provided herein, R¹ is —(CH₂)₂—NH—(CH₂)₉—CH₃ and the bacterial infection is Propionibacterium acnes (sldn acne), Eggerthella lenta (bacteremia) or Peptostreptococcus anaerobius (gynecological infection). In a further embodiment, R⁴ is diethanolamine or an amino acid. The amino acid, in one embodiment, is D-alanine, β-alanine, aspartic acid, glutamic acid, glycine or iminodiacetic acid.

In one embodiment, the bacterial infection is a methicillin-resistant Staphylococcus aureus (MRSA) infection and the composition administered to the patient in need thereof comprises an effective amount of a compound of Formula (I), Formula (II), or a pharmaceutically acceptable salt of Formula (I) or Formula (II), wherein R¹ is —(CH₂)₂—NH—(CH₂)₉—CH₃ and R⁴ is an amino acid or peptide. In a further embodiment, the administration is via a nebulizer or a dry powder inhaler and the bacterial infection is a pulmonary infection. In another embodiment, administration of a compound of Formula (I) is intravenous, R¹ is —(CH₂)₂—NH—(CH₂)₉—CH₃; R² is OH and R³ and R⁴ are H. In a further embodiment, X is O.

### BRIEF DESCRIPTION OF THE FIGURES

FIG. 1, top shows the reductive amination of vancomycin to arrive at a glycopeptide derivative. The reaction occurs at the primary amine of vancomycin. FIG. 1, bottom, shows a synthesis scheme for a chloroeremomycin derivative.

FIG. 2 shows synthesis schemes for making the glycopeptide derivative RV40 and its lactate salt.

FIG. 3 shows a synthesis scheme for making the glycopeptide derivative RV79.

FIG. 4 is a synthesis scheme for making alkyl vancomycin derivatives.

FIG. 5 shows one synthesis scheme for making decyl-vancomycin (Compound #5).

FIG. 6 is a graph of glycopeptide mass in rat lung, normalized to glycopeptide mass IPD, as a function of time. IPD: Immediate post dose (0.5 h).

### DETAILED DESCRIPTION OF THE INVENTION

The high frequency of multidrug resistant bacteria, and in particular, Gram-positive bacteria, both in the healthcare setting and the community present a significant challenge for the management of infections (Krause et al. (2008). Antimicrobial Agents and Chemotherapy 52(7), pp. 2647-2652, incorporated by reference herein in its entirety for all purposes). Moreover, methicillin resistant S. aureus (MRSA) infections in cystic fibrosis (CF) patients is a concern, and there is a lack of clinical data regarding approaches to eradicate such infections (Goss and Muhlebach (2011). Journal of Cystic Fibrosis 10, pp. 298-306, incorporated by reference herein in its entirety for all purposes).

The present invention addresses the need for new bacterial infection treatment methods, and in particular, bacterial infection treatment methods by delivering compounds of Formula (I), Formula (II), or a pharmaceutically acceptable salt of Formula (I) or Formula (II) to patients in need thereof, for example via the pulmonary or intravenous route.

In one aspect, the present invention relates to methods for treating bacterial infections, for example, Gram-positive bacterial infections and in some embodiments, Gram-positive bacterial pulmonary infections. The method, in one embodiment, comprises administering to a patient in need thereof, a composition comprising an effective amount of a compound of Formula (I), Formula (II), or a pharmaceutically acceptable salt of Formula (I) or Formula (II). The composition can be administered by any route. In the case of a pulmonary infection, in one embodiment, the composition is administered via a nebulizer, dry powder inhaler or metered dose inhaler. In another embodiment, the composition is administered intravenously.

The compounds for use in the bacterial infection treatment methods, and the specific treatment methods, are discussed in detail below.

An “effective amount” of a compound of Formula (I), Formula (II), or a pharmaceutically acceptable salt of Formula (I) or Formula (II), is an amount that can provide the desired therapeutic response. The effective amount can refer to a single dose as part of multiple doses during an administration period, or as the total dosage of glycopeptide given during an administration period. A treatment regimen can include substantially the same dose for each glycopeptide administration, or can comprise at least one, at least two or at least three different dosages.

The term “alkyl” refers to a monoradical branched or unbranched saturated hydrocarbon chain having from 1 to 40 carbon atoms, e.g., from 1 to 10 carbon atoms, or from 1 to 6 carbon atoms. This term is exemplified by groups such as methyl, ethyl, n-propyl, iso-propyl, n-butyl, iso-butyl, n-hexyl, n-decyl, tetradecyl, and the like. Both linear and branched alkyl groups are encompassed by the term “alkyl”.

The term “substituted alkyl” refers to an alkyl group as defined above, having from 1 to 8 substituents, e.g., from 1 to 5 substituents or from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkylene” refers to a diradical of a branched or unbranched saturated hydrocarbon chain, for example, having from 1 to 40 carbon atoms, e.g., from 1 to 10 carbon atoms, or from 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CCH₂—), the butylene isomers (e.g., —CH₂CH₂CH₂CH₂—) and the like.

The term “substituted alkylene” refers to an alkylene group, as defined above, having from 1 to 5 substituents, for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl. Additionally, such substituted alkylene groups include those where 2 substituents on the alkylene group are fused to form one or more cycloalkyl, substituted cycloalkyl, cycloalkenyl, substituted cycloalkenyl, aryl, heterocyclic or heteroaryl groups fused to the alkylene group. Such fused groups can contain from 1 to 3 fused ring structures. Additionally, the term substituted alkylene includes alkylene groups in which from 1 to 5 of the alkylene carbon atoms are replaced with oxygen, sulfur or NR— where R is hydrogen or alkyl. Examples of substituted alkylenes are chloromethylene (—CH(Cl)—), aminoethylene (—CH(NH₂)CH₂—), 2-carboxypropylene isomers (—CH₂CH(CO₂H)CH₂—), ethoxyethyl (—CH₂CH₂—O—CH₂CH₂—) and the like.

The term “alkaryl” refers to the groups -alkylene-aryl and substituted alkylene-aryl where alkylene, substituted alkylene and aryl are defined herein. Such alkaryl groups are exemplified by benzyl, phenethyl and the like.

The term “alkoxy” refers to the groups alkyl-O—, alkenyl-O—, cycloalkyl-O-cycloalkenyl-O—, and alkynyl-O—, where alkyl, alkenyl, cycloalkyl, cycloalkenyl, and alkynyl are as defined herein. Alkyl-O— alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkylalkoxy” refers to the groups -alkylene-O-alkyl, alkylene-O-substituted alkyl, substituted alkylene-O-alkyl and substituted alkylene-O-substituted alkyl wherein alkyl, substituted alkyl, alkylene and substituted alkylene are as defined herein. Alkylalkoxy groups are also expressed as alkylene-O-alkyl and include, by way of example, methylenemethoxy (—CH₂OCH₃), ethylenemethoxy (—CH₂CH₂OCH₃), n-propylene-iso-propoxy (—CH₂CH₂CH₂OCH(CH₃)₂), methylene-t-butoxy (—CH₂—O—C(CH₃)₃) and the like.

The term “alkenyl” refers to a monoradical of a branched or unbranched unsaturated hydrocarbon group having from 2 to 40 carbon atoms, e.g., 2 to 10 carbon atoms or 2 to 6 carbon atoms, and having at least 1 and in some embodiments, from 1-6 sites of vinyl unsaturation. Alkenyl groups include ethenyl (—CH═CH₂), n-propenyl (—CH₂CH═CH₂), iso-propenyl (—C(CH₃)═CH₂), and the like.

The term “substituted alkenyl” refers to an alkenyl group as defined above having from 1 to 5 substituents, and e.g., from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkenylene” refers to a diradical of a branched or unbranched unsaturated hydrocarbon group having from 2 to 40 carbon atoms, for example from 2 to 10 carbon atoms or from 2 to 6 carbon atoms and having at least 1 and for example, from 1-6 sites of vinyl unsaturation. This term is exemplified by groups such as ethenylene (—CH═CH—), the propenylene isomers (e.g., —CH₂CH═CH— and —C(CH₃)═CH—) and the like.

The term “substituted alkenylene” refers to an alkenylene group as defined above having from 1 to 5 substituents, and for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl. Additionally, such substituted alkenylene groups include those where 2 substituents on the alkenylene group are fused to form one or more cycloalkyl, substituted cycloalkyl, cycloalkenyl, substituted cycloalkenyl, aryl, heterocyclic or heteroaryl groups fused to the alkenylene group.

The term “alkynyl” refers to a monoradical of an unsaturated hydrocarbon having from 2 to 40 carbon atoms, for example, from 2 to 20 carbon atoms, or from 2 to 6 carbon atoms and having at least 1 and in some embodiments from 1 to 6 sites of acetylene (triple bond) unsaturation. Representative alkynyl groups include ethynyl (—C≡CH), propargyl (—CH₂C≡CH) and the like.

The term “substituted alkynyl” refers to an alkynyl group as defined above having from 1 to 5 substituents, for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkynylene” refers to a diradical of an unsaturated hydrocarbon having from 2 to 40 carbon atoms, for example from 2 to 10 carbon atoms or 2 to 6 carbon atoms and having at least 1 and in some embodiment, from 1-6 sites of acetylene (triple bond) unsaturation. Representative alkynylene groups include ethynylene (—C≡C—), propargylene (—CH₂C≡C—).

The term “substituted alkynylene” refers to an alkynylene group as defined above having from 1 to 5 substituents, for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO— aryl, —SO-heteroaryl, —SO₂-alkyl, —SO₂-substituted alkyl, —SO₂-aryl and —SO₂-heteroaryl.

The term “acyl” refers to the groups HC(O)—, alkyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)— where alkyl, substituted alkyl, cycloalkyl, substituted cycloalkyl, cycloalkenyl, substituted cycloalkenyl, aryl, heteroaryl and heterocyclic are as defined herein.

The term “acylamino” or “aminocarbonyl” refers to the group —C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” or “alkoxycarbonylamino” refers to the group —NRC(O)OR where each R is independently hydrogen, alkyl, substituted alkyl aryl, heteroaryl, or heterocyclic.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Representative aryls include phenyl, naphthyl and the like. Unless otherwise constrained by the definition for the aryl substituent, such aryl groups can optionally be substituted with from 1 to 5 substituents, e.g., from 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, sulfonamide, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. In one embodiment, the aryl substituent is alkyl, alkoxy, halo, cyano, nitro, trihalomethyl, thioalkoxy or a combination thereof.

The term “aryloxy” refers to the group aryl-O— wherein the aryl group is as defined above including optionally substituted aryl groups as also defined above.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic provided that both R groups are not H.

The term “carboxyalkyl” or “alkoxycarbonyl” refers to the groups “—C(O)O-alkyl”, “—C(O)O-substituted alkyl”, “—C(O)O-cycloalkyl”, “—C(O)O-substituted cycloalkyl”, “—C(O)O-alkenyl”, “—C(O)O-substituted alkenyl”, “—C(O)O-alkynyl” and “—C(O)O-substituted alkynyl” where alkyl, substituted alkyl, cycloalkyl, substituted cycloalkyl, alkenyl, substituted alkenyl, alkynyl and substituted alkynyl are as defined herein

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, and for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “cycloalkenyl” refers to cyclic alkenyl groups of from 4 to 20 carbon atoms having a single cyclic ring and at least one point of internal unsaturation. Examples of suitable cycloalkenyl groups include, e.g., cyclobut-2-enyl, cyclopent-3-enyl, cyclooct-3-enyl.

The term “substituted cycloalkenyl” refers to cycloalkenyl groups having from 1 to 5 substituents, and for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “halo” or “halogen” refers to fluoro, chloro, bromo and/or iodo.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” refers to an aromatic group of from 1 to 15 carbon atoms and 1 to 4 heteroatoms selected from oxygen, nitrogen and sulfur within at least one ring moiety.

Unless otherwise constrained by the definition for the heteroaryl substituent, such heteroaryl groups can be optionally substituted with 1 to 5 substituents, for example from 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, SO-alkyl, —SO-substituted alkyl, —SO-aryl, —SO-heteroaryl, —SO₂-alkyl, —SO₂-substituted alkyl, —SO₂-aryl and —SO₂-heteroaryl and trihalomethyl. Representative aryl substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). In one embodiment, the heteroaryl is pyridyl, pyrrolyl or furyl. “Heteroarylalkyl” refers to (heteroaryl)alkyl- where heteroaryl and alkyl are as defined herein. Representative examples include 2-pyridylmethyl and the like.

The term “heteroaryloxy” refers to the group heteroaryl-O—.

The term “heteroarylene” refers to the diradical group derived from heteroaryl (including substituted heteroaryl), as defined above, and is exemplified by the groups 2,6-pyridylene, 2,4-pyridiylene, 1,2-quinolinylene, 1,8-quinolinylene, 1,4-benzofuranylene, 2,5-pyridnylene, 2,5-indolenyl and the like.

The term “heterocycle” or “heterocyclic” refers to a monoradical saturated unsaturated group having a single ring or multiple condensed rings, from 1 to 40 carbon atoms and from 1 to 10 hetero atoms, for example from 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, and for example, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, SO-alkyl, —SO-substituted alkyl, —SO-aryl, —SO-heteroaryl, —SO₂-alkyl, —SO₂-substituted alkyl, —SO₂-aryl and —SO₂-heteroaryl. Such heterocyclic groups can have a single ring or multiple condensed rings. In one embodiment, the heterocyclic is morpholino or piperidinyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

Another class of heterocyclics is known as “crown compounds” which refers to a specific class of heterocyclic compounds having one or more repeating units of the formula [(CH₂-)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. In one embodiment, the crown compound has from 4 to 10 heteroatoms and 8 to 40 carbon atoms.

The term “heterocyclooxy” refers to the group heterocyclic-O—.

The term “heterocyclene” refers to the diradical group formed from a heterocycle, as defined herein, and is exemplified by the groups 2,6-morpholino, 2,5-morpholino and the like.

The term “oxyacylamino” or “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “spiro-attached cycloalkyl group” refers to a cycloalkyl group attached to another ring via one carbon atom common to both rings.

The term “sulfonamide” refers to a group of the formula —SO₂NRR, where each R is independently hydrogen, alkyl, substituted alkyl, aryl, heteroaryl, or heterocyclic wherein alkyl, substituted alkyl, aryl, heteroaryl and heterocyclic are as defined herein.

The term “thiol” refers to the group —SH.

The term “thioheteroaryloxy” refers to the group heteroaryl-S— wherein the heteroaryl group is as defined above including optionally substituted aryl groups as also defined above.

As to any of the above groups which contain one or more substituents, it is understood that such groups do not contain any substitution or substitution patterns which are sterically impractical and/or synthetically non-feasible. In addition, the compounds of this invention include all stereochemical isomers arising from the substitution of these compounds.

“Glycopeptide” refers to heptapeptide antibiotics, characterized by a multi-ring peptide core optionally substituted with saccharide groups. Examples of glycopeptides included in this definition may be found in “Glycopeptides Classification, Occurrence, and Discovery”, by Raymond C. Rao and Louise W. Crandall, (“Drugs and the Pharmaceutical Sciences” Volume 63, edited by Ramakrishnan Nagarajan, published by Marcal Dekker, Inc.), which is hereby incorporated by reference in its entirety. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimycin, Chloroorientiein, Chloropolysporin, Decaplanin, N-demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, Telavancin, UK-68597, UK-69542, UK-72051, Vancomycin, and the like. The term “glycopeptide” as used herein is also intended to include the general class of peptides disclosed above on which the sugar moiety is absent, i.e., the aglycone series of glycopeptides. For example, removal of the disaccharide moiety appended to the phenol on vancomycin by mild hydrolysis gives vancomycin aglycone. Also within the scope of the invention are glycopeptides that have been further appended with additional saccharide residues, especially aminoglycosides, in a manner similar to vancosamine. In embodiments described herein, one or more of the aforementioned glycopeoptides can be used in combination with a compound of Formula (I), Formula (II), or a pharmaceutically acceptable salt of Formula (I) or (II).

“Pharmaceutically acceptable salt” includes both acid and base addition salts. A pharmaceutically acceptable addition salt refers to those salts which retain the biological effectiveness and properties of the free bases, which are not biologically or otherwise undesirable, and which are formed with inorganic acids such as, but are not limited to, hydrochloric acid (HCl), hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid (e.g., as lactate), lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, acetic acid (e.g., as acetate), tartaric acid, thiocyanic acid, p-toluenesulfonic acid, trifluoroacetic acid (TFA), undecylenic acid, and the like. In one embodiment, the pharmaceutically acceptable salt is HCl, TFA, lactate or acetate.

A pharmaceutically acceptable base addition salt retains the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Inorganic salts include the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethyl pi peri dine, polyamine resins and the like. Organic bases that can be used to form a pharmaceutically acceptable salt include isopropylamine, diethylamine, ethanolamine, trimethylamine, dicyclohexylamine, choline and caffeine.

“Amino acid” refers to any of the naturally occurring amino acids, synthetic amino acids, and derivatives thereof, α-Amino acids comprise a carbon atom to which is bonded an amino group, a carboxy group, a hydrogen atom, and a distinctive group referred to as a “side chain”. The side chains of naturally occurring amino acids are well known in the art and include, for example, hydrogen (e.g., glycine), alkyl (e.g., alanine, valine, leucine, isoleucine, proline), substituted alkyl (e.g., as in threonine, serine, methionine, cysteine, aspartic acid, asparagine, glutamic acid, glutamine, arginine, and lysine), alkaryl (e.g., phenylalanine and tryptophan), substituted arylalkyl (e.g., tyrosine), and heteroarylalkyl (e.g., histidine).

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Gutamic acid; G=Gly=Glycine; H=His=Histidine; I=Ile=lsoleucine; L=Leu=Leucine; K=Lys=Lysine; M=Met=Methionine; F=Phe=Phenylalanine; P=Pro=Proline; S=Ser=Serine; T=Thr=Threonine; W=Trp=Tryptophan; Y=Tyr=Tyrosine; V=Val=Valine. The amino acids in the compositions provided herein are L- or D-amino acids. In one embodiment, a synthetic amino acid is used in the compositions provided herein. In one embodiment, the amino acid increases the half-life, efficacy and/or bioavailability of the glycopeptide antibiotic in the composition. In a further embodiment, the glycopeptide antibiotic is vancomycin.

Amino acid derivatives are encompassed by the amino acids described herein and refer to moieties having both an amine functional group, either as NH₂, NHR, or NR₂, and a carboxylic acid functional group, either as NH₂, NHR, or NR₂, and a carboxylic acid functional group. The term “amino acids” encompasses both natural and unnatural amino acids, and can refer to alpha-amino acids, beta-amino acids, or gamma amino acids. Unless specified otherwise, an amino acid structure referred to herein can be any possible stereoisomer, e.g., the D or L enantiomer. In some embodiments, the amino acid derivatives are short peptides, including dipeptides and tripeptides. Exemplary amino acids and amino acid derivatives suitable for the invention include alanine (ALA), D-alanine (D-ALA), alanine-alanine (ALA-ALA), β-alanine (βALA), alanine-β-alanine (ALA-βALA), 3-aminobutanoic acid (3-ABA), gamma-aminobutyric acid (GABA), glutamic acid (GLU or GLUt), D-glutamic acid (D-GLU), glycine (GLY), glycylglycine (GLY-GLY), glycine-alanine (GLY-ALA), alanine-glycine (ALA-GLY), aspartic acid (ASP), D-aspartic acid (D-ASP), lysine-alanine-alanine (LYS-ALA-ALA), L-Lysine-D-alanine-D-alanine (L-LYS-D-ALA-D-ALA), bicine, tricine, sarcosine, and iminodiacetic acid (IDAA). Amino acids and derivatives thereof can be synthesized according to known techniques, or can be purchased from suppliers, e.g., Sigma-Aldrich (Milwaukee, Wis.).

In one aspect, a compound of Formula (I), or a pharmaceutically acceptable salt thereof is provided. The compound in one embodiment, is administered to a patient in need of treatment of a bacterial infection.

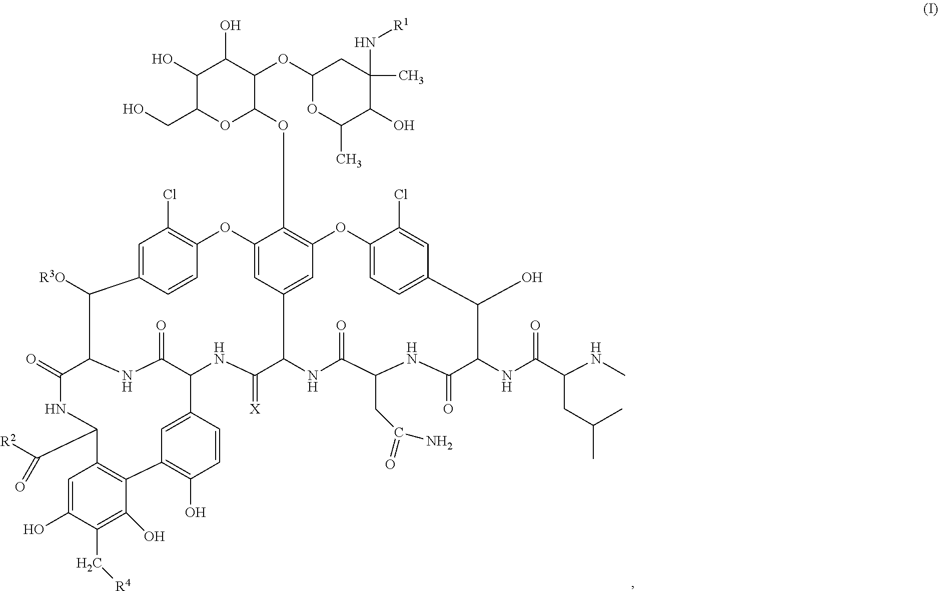

wherein,

R¹ is C₁-C₁₈ linear alkyl, C₁-C₁₈ branched alkyl, R⁵—Y—R⁶—(Z)ₙ, or

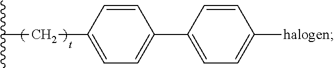

R² is —OH or —NH—(CH₇)_{q}—R⁷;

R₃ is H or

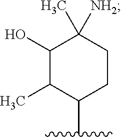 [SMILES: *C1CCC(C)(N)C(O)C1C]

R⁴ is diethanolamine, a monosaccharide, disaccharide, amino acid, or peptide, wherein the peptide has from 2 to 5 amino acids;

n is 1 or 2;

q is 1, 2, 3, 4, or 5;

t is 1, 2, 3, 4, or 5;

X is O, S, NH or H₂;

each Z is, independently, hydrogen, aryl, cycloalkyl, cycloalkenyl, heteroaryl or heterocycl;

R⁵ and R⁶ are independently selected from the group consisting of alkylene, alkenylene and alkynylene, wherein the alkylene, alkenylene and alkynylene groups are optionally substituted with from 1 to 3 substituents selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl

R⁷ is —N(CH₂)₂; —N⁺(CH₂)₃; or

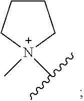 [SMILES: *[N+]1(C)CCCC1]

Y is oxygen, sulfur, —S—S—, —NR⁸—, —S(O)—, —SO₂—, —NR⁸C(O)—, —OSO₂—, —OC(O)—, —NR⁸SO₂—, —C(O)NR⁸—, —C(O)O—, —SO₂NR⁸—, —SO₂O—, —P(O)(OR⁸)O—, —P(O)(OR⁸)NR⁸—, —OP(O)(OR⁸)O—, —OP(O)(OR⁸)NR⁸—, —OC(O)O—, —NR⁸C(O)O—, —NR⁸C(O)NR⁸—, —OC(O)NR⁸— or —NR⁸SO₂NR⁸—; and

each R⁸ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, heteroaryl and heterocyclic.

Another aspect of the invention relates to a compound of Formula (II), or a pharmaceutically acceptable salt thereof:

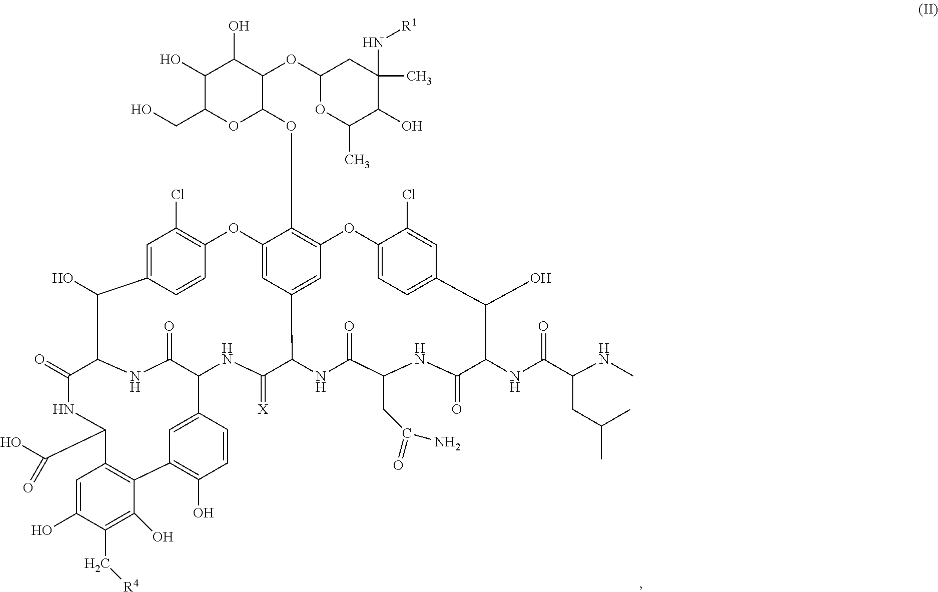

wherein,

R¹ is C₁-C₁₈ linear alkyl, C₁-C₁₈ branched alkyl, R⁵—Y—R⁶—(Z)ₙ, or

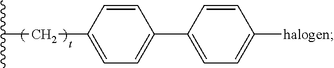

R⁴ is diethanolamine, a monosaccharide, disaccharide, amino acid, or peptide, wherein the peptide has from 2 to 5 amino acids;

n is 1 or 2; and

t is 1, 2, 3, 4, or 5;

X is O, S, NH or H₂.

each Z is, independently, hydrogen, aryl, cycloalkyl, cycloalkenyl, heteroaryl or heterocyclic;

R⁵ and R⁶ are independently selected from the group consisting of alkylene, alkenylene and alkynylene, wherein the alkylene, alkenylene and alkynylene groups are optionally substituted with from 1 to 3 substituents selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl;

Y is oxygen, sulfur, —S—S—, —NR⁸—, —S(O)—, —SO₂—, —OSO₂—, —NR⁸SO₂—, —SO₂NR⁸—, —SO₂O—, —P(O)(OR⁸)O—, —P(O)(OR⁸)NR⁸—, —OP(O)(OR⁸)O—, —OP(O)(OR⁸)NR⁸—, —NR⁸C(O)NR⁸—, or —NR⁸SO₂NR⁸—; and

each R⁸ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, heteroaryl and heterocyclic.

Compounds of Formula (I) and Formula (II) are synthesized, in one embodiment, by the methods provided in U.S. Pat. Nos. 6,455,669 and/or 7,160,984, the disclosure of each of which is incorporated by reference herein in their entireties. Further synthesis methods are provided in the Example section, herein. Other preparation steps and methods that can be employed are disclosed in U.S. Pat. No. 6,392,012; U.S. Patent Application Publication No. 2017/0152291; U.S. Patent Application Publication No. 2016/0272682, each of which is hereby incorporated by reference in their entirety for all purposes. Methods described in International Publication No. WO 2018/08197, the disclosure of which is incorporated by reference in its entirety, can also be employed. Synthesis schemes are also provided at the Example section, herein.

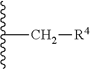

can be added to the resorcinol ring of a glycopeptide via Mannich reaction, for example, as described in Guan et al. (2018). J. Med. Chem. 61, pp. 286, 304; or Pavlov et al. (1997) The Journal of Antibiotics 50(6), pp. 509-513, each of which is incorporated by reference herein in its entirety.

As provided above, a

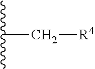

group at the resorcinol moiety of a glycopeptide, such as vancomycin, can be introduced via a Mannich reaction. Such reactions are described in for example, Guan et al. (2018). J. Med. Chem. 61, pp. 286, 304; or Pavlov et al. (1997) The Journal of Antibiotics 50(6), pp. 509-513, and U.S. Pat. No. 6,635,618, each of which is incorporated by reference herein in its entirety. In this reaction, an amine of formula NHRR′ (e.g., an amino acid, diethanoloamine, or a compound wherein one or both of R and R′ is a group that comprises a monosaccharide or disaccharide), and formaldehyde or formalin (a source of formaldehyde), are reacted with the glycopeptide under basic conditions to give the glycopeptide derivative having the

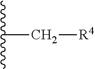

group.

In one embodiment, compounds of Formula (I) and Formula (II), e.g., where R¹ is

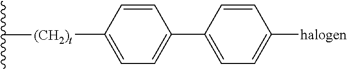

and R² is OH, are synthesized according to the methods provided in U.S. Patent Application Publication No. 2017/0152291, the disclosure of which is incorporated by reference in its entirety.

In embodiments of Formula (I) where R² is —NH—(CH₂)_{q}—R⁷, the amide coupling can be carried out as described in Yarlagadda et al. (2014). J. Med Chem. 57, pp. 4558-4568, the disclosure of which is incorporated by reference herein in its entirety for all purposes. For example, a solution of vancomycin or other glycopeptide derivative (e.g., a compound of Formula (I) where R¹ is

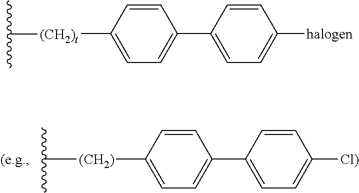

and X is O) can be treated with a solution of —NH—(CH₂)_{q}—R⁷ (e.g., a solution of —NH—(CH₂)₃—N(CH₂)₂, —NH—(CH₂)₃—N⁺(CH₂)₃, or

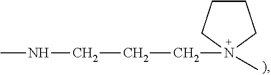

N-methyl morpholine and HBTU at 25° C. The reaction mixture can be stirred at 25° C. for 5 min and quenched with the addition of 50% MeOH in H₂O at 25° C. The mixture can be purified by semi-preparative reverse-phase HPLC to afford the compound as a white film.

In one embodiment, of a compound of Formula (I), Formula (II), or a pharmaceutically acceptable salt of Formula (I) or Formula (II), R¹ does not include a physiologically cleavable functional group. Stated another way, the R¹ group, in one embodiment, is not subject to hydrolysis or enzymatic cleavage in vivo.

In another embodiment, R¹ does not include an amide or ester moiety.

In one embodiment, a compound of Formula (I), Formula (II), or a pharmaceutically acceptable salt of Formula (I) or Formula (II) is provided, where R¹ is R⁵—Y—R⁶—(Z)ₙ. In a further embodiment, R⁵ is —(CH₂)₂—, R⁶ is —(CH₂)₁₀—, X is O, Y is NR⁸, Z is hydrogen and n is 1. In a further embodiment, R⁸ is hydrogen. As such, one embodiment of the method provided herein includes delivering to a patient a composition comprising an effective amount of a compound of Formula (I), Formula (II), or a pharmaceutically acceptable salt of Formula (I) or Formula (II), where R¹ is —(CH₂)₂—NH—(CH₂)₉—CH₃. In a further embodiment, X is O, R² is OH and R³ and R⁴ are H (for compounds of Formula (I)). In even a further embodiment, administration is via the intravenous or pulmonary route. In a further embodiment, R⁴ is diethanolamine or an amino acid. The amino acid, in one embodiment, is D-alanine, β-alanine, aspartic acid, glutamic acid, glycine or iminodiacetic acid.

In one embodiment, R⁴ is a monosaccharide. For example, the monosaccharide can be attached to the glycopeptide resorcinol ring via a Mannich reaction. As such, R⁴, in one embodiment, can be selected from one of the following structures:

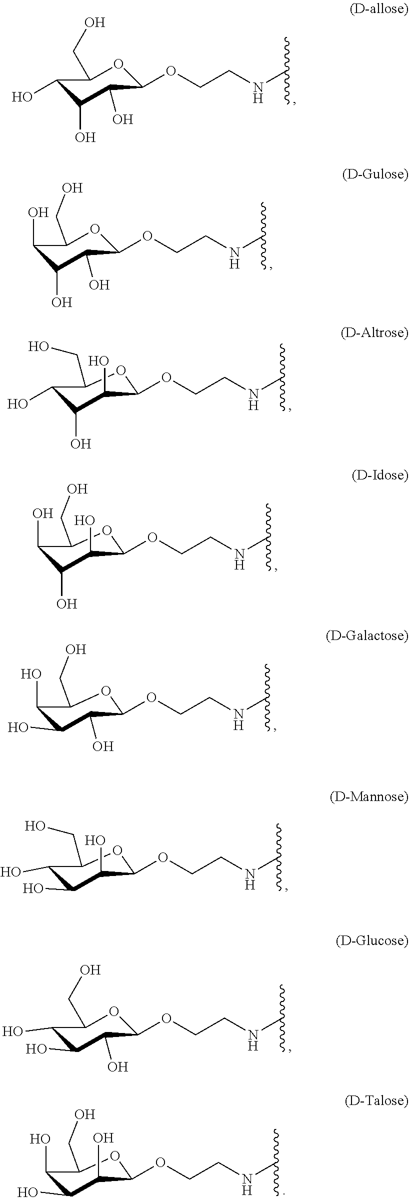

In a further embodiment, R¹ is —(CH₂)₂—NH—(CH₂)₉—CH₃. In even a further embodiment, X is O.

In one embodiment of a compound of Formula (I), Formula (II), or a pharmaceutically acceptable salt of Formula (I) or Formula (II), R¹ is —CH₂—NH—(CH₂)₁₀—CH₃. In a further embodiment, X is O, R² is OH and R³ and R⁴ are H. In a further embodiment, R⁴ is diethanolamine or an amino acid. The amino acid, in one embodiment, is D-alanine, β-alanine, aspartic acid, glutamic acid, glycine or iminodiacetic acid.

In one embodiment of a compound of Formula (I), Formula (II), or a pharmaceutically acceptable salt thereof, R¹ is —(CH₂)₂—NH—(CH₂)₁₀—CH₃. In a further embodiment, X is O. In a further embodiment, R⁴ is diethanolamine or an amino acid. The amino acid, in one embodiment, is D-alanine, β-alanine, aspartic acid, glutamic acid, glycine or iminodiacetic acid.

In another embodiment of a compound of Formula (I), Formula (II), or a pharmaceutically acceptable salt thereof, R¹ is —(CH₂)₂—NH—(CH₂)₁₁—CH₃. In a further embodiment, X is O, R² is OH and R³ and R⁴ are H.

In another embodiment, a compound of Formula (I), or a pharmaceutically acceptable salt of Formula (I), R¹ is

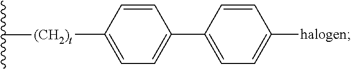

X is O or H₂; and R² is —NH—(CH₂)_{q}—R⁷. In a further embodiment, R² is —NH—(CH₂)₃—R⁷. In a further embodiment, R¹ is

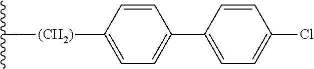

and R⁷ is —N⁺(CH₂)₃ or —N(CH₂)₂. In a further embodiment, R⁴ is diethanolamine or an amino acid. The amino acid, in one embodiment, is D-alanine, β-alanine, aspartic acid, glutamic acid, glycine or iminodiacetic acid.

In yet another embodiment, R¹ is C₁₀-C₁₆ alkyl. In even a further embodiment, R¹ is C₁₀ alkyl.

In yet another embodiment of a compound of Formula (I), Formula (II), or a pharmaceutically acceptable salt of Formula (I), R² is OH, R³ and R⁴ are H and X is O. In a further embodiment, R¹ is

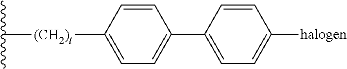

or R⁵—Y—R⁶—(Z)ₙ. In even a further embodiment, R¹ is R⁵—Y—R⁶—(Z)ₙ, R⁵ is methylene, ethylene or propylene; R⁶ is —(CH₂)₉—, —(CH₂)₁₀—, —(CH₂)₁₁—, or —(CH₂)₁₂—, Z is H and n is 1. In a further embodiment, R⁴ is diethanolamine or an amino acid. The amino acid, in one embodiment, is D-alanine, β-alanine, aspartic acid, glutamic acid, glycine or iminodiacetic acid.

In yet another embodiment of a compound of Formula (I), Formula (II), or a pharmaceutically acceptable salt thereof, one or more hydrogen atoms is replaced with a deuterium atom.

In one embodiment of a compound of Formula (I), Formula (II), or a pharmaceutically acceptable salt of Formula (I) or Formula (II), R¹ is R⁵—Y—R⁶—(Z)ₙ. In a further embodiment, R⁵ is —(CH₂)₂—, R⁶ is —(CH₂)₁₀—, Y is NR⁸, Z is hydrogen and n is 1. In a further embodiment, R⁸ is hydrogen. In a further embodiment, R⁴ is diethanolamine or an amino acid. The amino acid, in one embodiment, is D-alanine, β-alanine, aspartic acid, glutamic acid, glycine or iminodiacetic acid.

In one embodiment, R¹ is —(CH₂)₂—NH—(CH₂)₉—CH₃. In a further embodiment, R⁴ is diethanolamine or an amino acid. The amino acid, in one embodiment, is D-alanine, β-alanine, aspartic acid, glutamic acid, glycine or iminodiacetic acid.

In yet another embodiment, a compound of Formula (I), Formula (II), or a pharmaceutically acceptable salt thereof, X is O, R¹ is R⁵—Y—R⁶—(Z)ₙ, R² is OH, and R³ is H.

In a further embodiment, R⁴ is diethanolamine or an amino acid. The amino acid, in one embodiment, is D-alanine, β-alanine, aspartic acid, glutamic acid, glycine or iminodiacetic acid.

In a further embodiment, R⁵ is —(CH₂)₂—, R⁶ is —(CH₂)₁₀—, Y is NR⁸, Z is hydrogen and n is 1. In a further embodiment, R⁸ is hydrogen and X is O. In even a further embodiment, the administering is intravenous or via the pulmonary route. In a further embodiment, R⁴ is diethanolamine or an amino acid. The amino acid, in one embodiment, is D-alanine, β-alanine, aspartic acid, glutamic acid, glycine or iminodiacetic acid.

In one embodiment of a compound of Formula (I) or a pharmaceutically acceptable salt thereof, R¹ is —(CH₂)₂—NH—(CH₂)₉—CH₃, X is O, R² is —NH—(CH₂)_{q}—R⁷, R³ is H and R⁴ is diethanolamine or an amino acid. The amino acid, in one embodiment, is D-alanine, β-alanine, aspartic acid, glutamic acid, glycine or iminodiacetic acid. In a further embodiment, q is 2 or 3 and R⁷ is —N(CH₂)₂.

In one embodiment, a compound of Formula (I) or a pharmaceutically acceptable salt thereof is provided, where R¹ is —(CH₂)₂—NH—(CH₂)₉—CH₃, X is O, R² is OH, R³ is

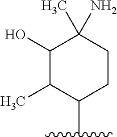 [SMILES: *C1CCC(C)(N)C(O)C1C]

and R⁴ an amino acid or diethanolamine. The amino acid, in one embodiment, is D-alanine, β-alanine, aspartic acid, glutamic acid, glycine or iminodiacetic acid.

In one embodiment of a compound of Formula (I) or a pharmaceutically acceptable salt thereof, R¹ is —(CH₂)₂—NH—(CH₂)₉—CH₃, X is O, R² is OH, and R³ is H and R⁴ is diethanolamine or an amino acid. The amino acid, in one embodiment, is D-alanine, β-alanine, aspartic acid, glutamic acid, glycine or iminodiacetic acid.

In yet another embodiment, a compound of Formula (I) or Formula (II) is provided, wherein one or more hydrogen atoms is replaced with a deuterium atom. In a further embodiment, R²—Y—R³—(Z)ₙ is —(CH₂)₂—NH—(CH₂)₉—CH₃.

In one embodiment of a compound of Formula (I), Formula (II), or a pharmaceutically acceptable salt of Formula (I) or Formula (II), R¹ is (CH₂)ₙ₁—Y—(CH₂)ₙ₂—CH₃, R² is OH, R³ and R⁴ are H, n1 is an integer selected from 1 to 6 and n2 is an integer from 1 to 15. In a further embodiment, X is O.

In one embodiment, a compound of Formula (I), Formula (II), or a pharmaceutically acceptable salt of Formula (I) or Formula (II), R¹ is (CH₂)—Y—(CH₂)ₙ₂—CH₃.

In a further embodiment, Y is oxygen, sulfur, —S—S—, —NH—, —S(O)— or —SO₂— and n2 is an integer from 5 to 10. In a further embodiment, Y is —NH—. In one embodiment, R⁴ is a monosaccharide, diethanolamine or an amino acid. The amino acid, in one embodiment, is D-alanine, β-alanine, aspartic acid, glutamic acid, glycine or iminodiacetic acid.

In one embodiment of a compound of Formula (I), or a pharmaceutically acceptable salt thereof, R¹ is (CH₂)₂—Y—(CH₂)ₙ₂—CH₃, R² is OH, R³ is H, X is O and n2 is an integer from 5 to 10. In a further embodiment, Y is oxygen, sulfur, —S—S—, —NH—, —S(O)— or —SO₂—. In a further embodiment, Y is —NH—. In a further embodiment, R⁴ is a monosaccharide, diethanolamine or an amino acid. The amino acid, in one embodiment, is D-alanine, β-alanine, aspartic acid, glutamic acid, glycine or iminodiacetic acid.

In one embodiment of a compound of Formula (I), Formula (II), or a pharmaceutically acceptable salt thereof, R¹ is (CH₂)₃—Y—(CH₂)ₙ₂—CH₃, X is O, and n2 is an integer from 5 to 10. In a further embodiment, Y is oxygen, sulfur, —S—S—, —NH—, —S(O)— or —SO₂—. In a further embodiment, Y is —NH—. In a further embodiment, R⁴ is a monosaccharide, diethanolamine or an amino acid. The amino acid, in one embodiment, is D-alanine, β-alanine, aspartic acid, glutamic acid, glycine or iminodiacetic acid.

In one embodiment of a compound of Formula (I), or a pharmaceutically acceptable salt thereof, R¹ is (CH₂)₁₋₃—Y—(CH₂)₈—CH₃, R² is OH, R³ is H and X is O. In a further embodiment, Y is oxygen, sulfur, —S—S—, —NH—, —S(O)— or —SO₂—. In a further embodiment, Y is —NH—. In a further embodiment, R⁴ is a monosaccharide, diethanolamine or an amino acid. The amino acid, in one embodiment, is D-alanine, β-alanine, aspartic acid, glutamic acid, glycine or iminodiacetic acid.

In one embodiment, a compound of Formula (I), or a pharmaceutically acceptable salt thereof, R¹ is (CH₂)₁₋₃—Y—(CH₂)₉—CH₃, R² is OH, R³ is H and X is O. In a further embodiment, Y is oxygen, sulfur, —S—S—, —NH—, —S(O)— or —SO₂—. In a further embodiment, Y is —NH—. In a further embodiment, R⁴ is a monosaccharide, diethanolamine or an amino acid. The amino acid, in one embodiment, is D-alanine, β-alanine, aspartic acid, glutamic acid, glycine or iminodiacetic acid.

In another embodiment, a compound of Formula (I), Formula (II), or a pharmaceutically acceptable salt of Formula (I) or Formula (II) is provided where R¹ is (CH₂)₂—Y—(CH₂)₁₀—CH₃, R² is OH, R³ and R⁴ are H and X is O. In a further embodiment, Y is oxygen, sulfur, —S—S—, —NH—, —S(O)— or —SO₂—. In a further embodiment, Y is —NH—. In a further embodiment, R⁴ is a monosaccharide, diethanolamine or an amino acid. The amino acid, in one embodiment, is D-alanine, β-alanine, aspartic acid, glutamic acid, glycine or iminodiacetic acid.

In another aspect of the invention, a composition is provided comprising an effective amount of a compound of Formula (I) or (II), or a pharmaceutically acceptable salt thereof. Compositions provided herein can be in the form of a solution, suspension or dry powder. Compositions can be administered by techniques known in the art, including, but not limited to intramuscular, intravenous, intratracheal, intranasal, intraocular, intraperitoneal, subcutaneous, and transdermal routes. In addition, as discussed throughout, the compositions can also be administered via the pulmonary route, e.g., via inhalation with a nebulizer or a dry powder inhaler.

In one embodiment, the composition provided herein comprises a plurality of nanoparticles of the antibiotic of Formula (I), Formula (II), or a pharmaceutically acceptable salt of Formula (I) or Formula (II) in association with a polymer. The mean diameter of the plurality of nanoparticles, in one embodiment, is from about 50 nm to about 900 nm, for example from about 10 nm to about 800 nm, from about 100 nm to about 700 nm, from about 100 nm to about 600 nm or from about 100 nm to about 500 nm.

In one embodiment, the plurality of nanoparticles comprise a biodegradable polymer and the antibiotic of Formula (I), Formula (II), or a pharmaceutically acceptable salt of Formula (I) or Formula (II). In a further embodiment, the biodegradable polymer is poly(D,L-lactide), poly(lactic acid) (PLA), poly(D,L-glycolide) (PLG), poly(lactide-co-glycolide) (PLGA), poly-(cyanoacrylate) (PCA), or a combination thereof.

In even a further embodiment, the biodegradable polymer is poly(lactic-co-glycolitic acid) (PLGA).

Nanoparticle compositions can be prepared according to methods known to those of ordinary skill in the art. For example, coacervation, solvent evaporation, emulsification, in situ polymerization, or a combination thereof can be employed (see, e.g., Soppimath et al. (2001). Journal of Controlled Release 70, pp. 1-20, incorporated by reference herein in its entirety for all purposes).

The amount of polymer in the composition can be adjusted, for example, to adjust the release profile of the compound of Formula from the composition.

In one embodiment, a dry powder composition disclosed in one of U.S. Pat. Nos. 5,874,064, 5,855,913 and/or U.S. Patent Application Publication No. 2008/0160092 is used to formulate one of the glycopeptides of Formula (I), Formula (II), or a pharmaceutically acceptable salt of Formula (I) or Formula (II). The disclosures of U.S. Pat. Nos. 5,874,064, 5,855,913 and U.S. Patent Application Publication No. 2008/0160092 are each incorporated by reference herein in their entireties for all purposes.

In one embodiment, the composition delivered via the methods provided herein are spray dried, hollow and porous particulate compositions. For example, the hollow and porous particulate compositions as disclosed in WO 1999/16419, hereby incorporated in its entirety by reference for all purposes, can be employed. Such particulate compositions comprise particles having a relatively thin porous wall defining a large internal void, although, other void containing or perforated structures are contemplated as well.

Compositions delivered via the methods provided herein, in one embodiment, yield powders with bulk densities less than 0.5 g/cm³ or 0.3 g/cm³, for example, less 0.1 g/cm3, or less than 0.05 g/cm³. By providing particles with very low bulk density, the minimum powder mass that can be filled into a unit dose container is reduced, which eliminates the need for carrier particles. Moreover, the elimination of carrier particles, without wishing to be bound by theory, can minimize throat deposition and any “gag” effect, since the large lactose particles can impact the throat and upper airways due to their size.

In some embodiments, the particulate compositions delivered via the methods provided herein comprise a structural matrix that exhibits, defines or comprises voids, pores, defects, hollows, spaces, interstitial spaces, apertures, perforations or holes. The particulate compositions in one embodiment, are provided in a “dry” state. That is, the particulate composition possesses a moisture content that allows the powder to remain chemically and physically stable during storage at ambient temperature and easily dispersible. As such, the moisture content of the microparticles is typically less than 6% by weight, and for example, less 3% by weight. In some embodiments, the moisture content is as low as 1% by weight. The moisture content is, at least in part, dictated by the formulation and is controlled by the process conditions employed, e.g., inlet temperature, feed concentration, pump rate, and blowing agent type, concentration and post drying.

Reduction in bound water can lead to improvements in the dispersibility and flowability of phospholipid based powders, leading to the potential for highly efficient delivery of powdered lung surfactants or particulate composition comprising active agent dispersed in the phospholipid.

The composition administered via the methods provided herein, in one embodiment, is a particulate composition comprising a compound of Formula (I) or Formula (II), a phospholipid and a polyvalent cation. In particular, the compositions of the present invention can employ polyvalent cations in phospholipid-containing, dispersible particulate compositions for pulmonary administration to the respiratory tract for local or systemic therapy via aerosolization.

Without wishing to be bound by theory, it is thought that the use of a polyvalent cation in the form of a hygroscopic salt such as calcium chloride stabilizes a dry powder prone to moisture induced stabilization. Without wishing to be bound by theory, it is thought that such cations intercalate the phospholipid membrane, thereby interacting directly with the negatively charged portion of the zwitterionic headgroup. The result of this interaction is increased dehydration of the headgroup area and condensation of the acyl-chain packing, all of which leads to increased thermodynamic stability of the phospholipids. Other benefits of such dry powder compositions are provided in U.S. Pat. No. 7,442,388, the disclosure of which is incorporated herein in its entirety for all purposes.

The polyvalent cation for use in the present invention in one embodiment, is a divalent cation. In a further embodiment, the divalent cation is calcium, magnesium, zinc or iron. The polyvalent cation is present in one embodiment, to increase the Tm of the phospholipid such that the particulate composition exhibits a Tm which is greater than its storage temperature Ts by at least 20° C. The molar ratio of polyvalent cation to phospholipid in one embodiment, is 0.05, e.g., from about 0.05 to about 2.0, or from about 0.25 to about 1.0. In one embodiment, the molar ratio of polyvalent cation to phospholipid is about 0.50. In one embodiment, the polyvalent cation is calcium and is provided as calcium chloride.

According to one embodiment, the phospholipid is a saturated phospholipid. In a further embodiment, the saturated phospholipid is a saturated phosphatidylcholine. Acyl chain lengths that can be employed range from about C₁₆ to C₂₂. For example, in one embodiment an acyl chain length of 16:0 or 18:0 (i.e., palmitoyl and stearoyl) is employed. In one phospholipid embodiment, a natural or synthetic lung surfactant is provided as the phospholipid component. In this embodiment, the phospholipid can make up to 90 to 99.9% w/w of the lung surfactant. Suitable phospholipids according to this aspect of the invention include natural or synthetic lung surfactants such as those commercially available under the trademarks ExoSurf, InfaSurf® (Ony, Inc.), Survanta, CuroSurf, and ALEC.

The Tm of the phospholipid-glycopeptide particles, in one embodiment, is manipulated by varying the amount of polyvalent cations in the composition.

Phospholipids from both natural and synthetic sources are compatible with the compositions administered by the methods provided herein, and may be used in varying concentrations to form the structural matrix. Generally compatible phospholipids comprise those that have a gel to liquid crystal phase transition greater than about 40° C. The incorporated phospholipids in one embodiment, are relatively long chain (i.e., C₁₆-C₂₂) saturated lipids and in a further embodiment, comprise saturated phospholipids. In even a further embodiment, the saturated phospholipid is a saturated phosphatidylcholine. In even a further embodiment, the saturated phosphatidylcholine has an acyl chain lengths of 16:0 or 18:0 (palmitoyl or stearoyl). Exemplary phospholipids useful in the disclosed stabilized preparations comprise, phosphoglycerides such as dipalmitoylphosphatidylcholine (DPPC), disteroylphosphatidylcholine (DSPC), diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, long-chain saturated phosphatidylinositols.

In addition to the phospholipid, a co-surfactant or combinations of surfactants, including the use of one or more in the liquid phase and one or more associated with the particulate compositions can be used in the compositions delivered via the methods provided herein. By “associated with or comprise” it is meant that the particulate compositions may incorporate, adsorb, absorb, be coated with or be formed by the surfactant. Surfactants include fluorinated and nonfluorinated compounds and can include saturated and unsaturated lipids, nonionic detergents, nonionic block copolymers, ionic surfactants and combinations thereof. In one embodiment comprising stabilized dispersions, nonfluorinated surfactants are relatively insoluble in the suspension medium.

Compatible nonionic detergents suitable as co-surfactants in the compositions provided herein include sorbitan esters including sorbitan trioleate (Span™ 85), sorbitan sesquioleate, sorbitan monooleate, sorbitan monolaurate, polyoxyethylene (20) (Brij® S20), sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Block copolymers include diblock and triblock copolymers of polyoxyethylene and polyoxypropylene, including poloxamer 188 (Pluronic® F-68), poloxamer 407 (Pluronic® F-127), and poloxamer 338. Ionic surfactants such as sodium sulfosuccinate, and fatty acid soaps may also be utilized.

The phospholipid-glycopeptide particulate compositions can include additional lipids such as a glycolipid, ganglioside GM1, sphingomyelin, phosphatidic acid, cardiolipin; a lipid bearing a polymer chain such as polyethylene glycol, chitin, hyaluronic acid, or polyvinylpyrrolidone; a lipid bearing sulfonated mono-, di-, and polysaccharides; a fatty acid such as palmitic acid, stearic acid, and/or oleic acid; cholesterol, cholesterol esters, and cholesterol hemisuccinate.

In addition to the phospholipid and polyvalent cation, the particulate composition delivered via the methods provided herein can also include a biocompatible, and in some embodiments, biodegradable polymer, copolymer, or blend or other combination thereof. The polymer in one embodiment is a polylactide, polylactide-glycolide, cyclodextrin, polyacrylate, methylcellulose, carboxymethylcellulose, polyvinyl alcohol, polyanhydride, polylactam, polyvinyl pyrrolidone, polysaccharide (e.g., dextran, starch, chitin, chitosan), hyaluronic acid, protein (e.g., albumin, collagen, gelatin, etc.).

Besides the aforementioned polymer materials and surfactants, other excipients can be added to a particulate composition, for example, to improve particle rigidity, production yield, emitted dose and deposition, shelf-life and/or patient acceptance. Such optional excipients include, but are not limited to: coloring agents, taste masking agents, buffers, hygroscopic agents, antioxidants, and chemical stabilizers. Other excipients may include, but are not limited to, carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Mixtures of carbohydrates and amino acids are further held to be within the scope of the present invention. The inclusion of both inorganic (e.g., sodium chloride), organic acids and their salts (e.g., carboxylic acids and their salts such as sodium citrate, sodium ascorbate, magnesium gluconate, sodium gluconate, tromethamine hydrochloride, etc.) and buffers can also be undertaken. Salts and/or organic solids such as ammonium carbonate, ammonium acetate, ammonium chloride or camphor can also be employed.

According to one embodiment, the particulate compositions may be used in the form of dry powders or in the form of stabilized dispersions comprising a non-aqueous phase. The dispersions or powders of the present invention may be used in conjunction with metered dose inhalers (MDIs), dry powder inhalers (DPIs), atomizers, or nebulizers to provide for pulmonary delivery.

While several procedures are generally compatible with making certain dry powder compositions described herein, spray drying is a particularly useful method. As is well known, spray drying is a one-step process that converts a liquid feed to a dried particulate form. With respect to pharmaceutical applications, it will be appreciated that spray drying has been used to provide powdered material for various administrative routes including inhalation. See, for example, M. Sacchetti and M. M. Van Oort in: Inhalation Aerosols: Physical and Biological Basis for Therapy, A. J. Hickey, ed. Marcel Dekkar, New York, 1996, which is incorporated herein by reference in its entirety for all purposes. In general, spray drying consists of bringing together a highly dispersed liquid, and a sufficient volume of hot air to produce evaporation and drying of the liquid droplets. The preparation to be spray dried or feed (or feed stock) can be any solution, suspension, slurry, colloidal dispersion, or paste that may be atomized using the selected spray drying apparatus. In one embodiment, the feed stock comprises a colloidal system such as an emulsion, reverse emulsion, microemulsion, multiple emulsion, particulate dispersion, or slurry. Typically, the feed is sprayed into a current of warm filtered air that evaporates the solvent and conveys the dried product to a collector. The spent air is then exhausted with the solvent.

It will further be appreciated that spray dryers, and specifically their atomizers, may be modified or customized for specialized applications, e.g., the simultaneous spraying of two solutions using a double nozzle technique. More specifically, a water-in-oil emulsion can be atomized from one nozzle and a solution containing an anti-adherent such as mannitol can be co-atomized from a second nozzle. In one embodiment, it may be desirable to push the feed solution though a custom designed nozzle using a high pressure liquid chromatography (HPLC) pump. Examples of spray drying methods and systems suitable for making the dry powders of the present invention are disclosed in U.S. Pat. Nos. 6,077,543, 6,051,256, 6,001,336, 5,985,248, and 5,976,574, each of which is incorporated in their entirety by reference for all purposes.

While the resulting spray-dried powdered particles typically are approximately spherical in shape, nearly uniform in size and frequently are hollow, there may be some degree of irregularity in shape depending upon the incorporated glycopeptide of Formula (I) or Formula (II) and the spray drying conditions. In one embodiment, an inflating agent (or blowing agent) is used in the spray-dried powder production, e.g., as disclosed in WO 99/16419, incorporated by reference herein in its entirety for all purposes. Additionally, an emulsion can be included with the inflating agent as the disperse or continuous phase. The inflating agent can be dispersed with a surfactant solution, using, for instance, a commercially available microfluidizer at a pressure of about 5000 to 15,000 PSI. This process forms an emulsion, and in some embodiments, an emulsion stabilized by an incorporated surfactant, and can comprise submicron droplets of water immiscible blowing agent dispersed in an aqueous continuous phase. The blowing agent in one embodiment, is a fluorinated compound (e.g., perfluorohexane, perfluorooctyl bromide, perfluorooctyl ethane, perfluorodecalin, perfluorobutyl ethane) which vaporizes during the spray-drying process, leaving behind generally hollow, porous aerodynamically light microspheres. Other suitable liquid blowing agents include nonfluorinated oils, chloroform, Freons, ethyl acetate, alcohols and hydrocarbons. Nitrogen and carbon dioxide gases are also contemplated as a suitable blowing agent. Perfluorooctyl ethane is the blowing agent, in one embodiment.

Whatever components are selected, the first step in particulate production in one embodiment, comprises feed stock preparation. The selected glycopeptide is dissolved in a solvent, for example water, dimethylformamide (DMF), dimethyl sulfoxide (DMSO), acetonitrile, ethanol, methanol, or combinations thereof, to produce a concentrated solution. The polyvalent cation may be added to the glycopeptide solution or may be added to the phospholipid emulsion as discussed below. The glycopeptide may also be dispersed directly in the emulsion, particularly in the case of water insoluble agents. Alternatively, the glycopeptide is incorporated in the form of a solid particulate dispersion. The concentration of the glycopeptide used is dependent on the amount of glycopeptide required in the final powder and the performance of the delivery device employed (e.g., the fine particle dose for a MDI or DPI). As needed, cosurfactants such as poloxamer 188 or span 80 may be dispersed into this annex solution. Additionally, excipients such as sugars and starches can also be added.

In one embodiment, a polyvalent cation-containing oil-in-water emulsion is then formed in a separate vessel. The oil employed in one embodiment, is a fluorocarbon (e.g., perfluorooctyl bromide, perfluorooctyl ethane, perfluorodecalin) which is emulsified with a phospholipid. For example, polyvalent cation and phospholipid may be homogenized in hot distilled water (e.g., 60° C.) using a suitable high shear mechanical mixer (e.g., Ultra-Turrax model T-25 mixer) at 8000 rpm for 2 to 5 minutes. In one embodiment, 5 to 25 g of fluorocarbon is added dropwise to the dispersed surfactant solution while mixing. The resulting polyvalent cation-containing perfluorocarbon in water emulsion is then processed using a high pressure homogenizer to reduce the particle size. In one embodiment, the emulsion is processed at 12,000 to 18,000 PSI, 5 discrete passes and kept at 50 to 80° C.

The glycopeptide solution (or suspension) and perfluorocarbon emulsion are then combined and fed into the spray dryer. In one embodiment, the two preparations are miscible. While the glycopeptide is solubilized separately for the purposes of the instant discussion it will be appreciated that, in other embodiments, the glycopeptide may be solubilized (or dispersed) directly in the emulsion. In such cases, the glycopeptide emulsion is simply spray dried without combining a separate glycopeptide preparation.

Operating conditions such as inlet and outlet temperature, feed rate, atomization pressure, flow rate of the drying air, and nozzle configuration can be adjusted in accordance with the manufacturer's guidelines in order to produce the desired particle size, and production yield of the resulting dry particles. The selection of appropriate apparatus and processing conditions are well within the purview of a skilled artisan. In one embodiment, the particulate composition comprises hollow, porous spray dried micro- or nano-particles.

Along with spray drying, particulate compositions useful in the present invention may be formed by lyophilization. Those skilled in the art will appreciate that lyophilization is a freeze-drying process in which water is sublimed from the composition after it is frozen. Methods for providing lyophilized particulates are known to those of skill in the art. The lyophilized cake containing a fine foam-like structure can be micronized using techniques known in the art.

Besides the aforementioned techniques, the glycopeptide particulate compositions or glycopeptide particles provided herein may be formed using a method where a feed solution (either emulsion or aqueous) containing wall forming agents is rapidly added to a reservoir of heated oil (e.g., perflubron or other high boiling FCs) under reduced pressure. The water and volatile solvents of the feed solution rapidly boils and are evaporated. In one embodiment, the wall forming agents are insoluble in the heated oil. The resulting particles can then separated from the heated oil using a filtering technique and then dried under vacuum.

In another embodiment, the particulate compositions of the present invention may also be formed using a double emulsion method. In the double emulsion method, the medicament is first dispersed in a polymer dissolved in an organic solvent (e.g., methylene chloride, ethyl acetate) by sonication or homogenization. This primary emulsion is then stabilized by forming a multiple emulsion in a continuous aqueous phase containing an emulsifier such as polyvinylalcohol. Evaporation or extraction using conventional techniques and apparatus then removes the organic solvent. The resulting particles are washed, filtered and dried prior to combining them with an appropriate suspension medium.

In order to maximize dispersibility, dispersion stability and optimize distribution upon administration, the mean geometric particle size of the particulate compositions in one embodiment, is from about 0.5-50 μm, for example from about 0.5 μm to about 10 μm or from about 0.5 to about 5 μm. In one embodiment, the mean geometric particle size (or diameter) of the particulate compositions is less than 20 μm or less than 10 μm. In a further embodiment, the mean geometric diameter is ≤about 7 μm or ≤5 μm. In even a further embodiment, the mass geometric diameter is ≤about 2.5 μm. In one embodiment, the particulate composition comprises a powder of dry, hollow, porous spherical shells of from about 0.1 to about 10 μm, e.g., from about 0.5 to about 5 μm in diameter, with shell thicknesses of approximately 0.1 μm to about 0.5 μm.

In addition to the glycopeptides of Formula (I), Formula (II) or a pharmaceutically acceptable salt thereof, one or more additional antiinfectives can be included in the composition administered to the patient in need thereof, either in the same composition, or a different composition. Additional antiinfectives include an additional glycopeptide, for example, one of the glycopeptides described herein. Other additional antiinfectives include but are not limited to aminoglycosides (e.g., dibekacin, K-4619, sisomicin, amikacin, dactimicin, isepamicin, rhodestreptomycin, apramycin, etimicin, KA-5685, sorbistin, arbekacin, framycetin, kanamycin, spectinomycin, astromicin, gentamicin, neomycin, sporaricin, bekanamycin, H107, netilmicin, streptomycin, boholmycin, hygromycin, paromomycin, tobramycin, brulamycin, hygromycin B, plazomicin, verdamicin, capreomycin, inosamycin, ribostamycin, vertilmicin), tetracyclines (e.g., chlortetracycline, oxytetracycline, methacycline, doxycycline, minocycline), sulfonamides (e.g., sulfanilamide, sulfadiazine, sulfamethaoxazole, sulfisoxazole, sulfacetamide), paraaminobenzoic acid, diaminopyrimidines (e.g., trimethoprim), quinolones (e.g., nalidixic acid, cinoxacin, ciprofloxacin and norfloxacin), penicillins (e.g., penicillin G, penicillin V, ampicillin, amoxicillin, bacampicillin, carbenicillin, carbenicillin indanyl, ticarcillin, azlocillin, mezlocillin, piperacillin), penicillinase resistant penicillin (e.g., methicillin, oxacillin, cloxacillin, dicloxacillin, nafcillin), first generation cephalosporins (e.g., cefadroxil, cephalexin, cephradine, cephalothin, cephapirin, cefazolin), second generation cephalosporins (e.g., cefaclor, cefamandole, cefonicid, cefoxitin, cefotetan, cefuroxime, cefuroxime axetil; cefmetazole, cefprozil, loracarbef, ceforanide), third generation cephalosporins (e.g., cefepime, cefoperazone, cefotaxime, ceftizoxime, ceftriaxone, ceftazidime, cefixime, cefpodoxime, ceftibuten), other β-lactams (e.g., imipenem, meropenem, aztreonam, clavulanic acid, sulbactam, tazobactam, and the like), betalactamase inhibitors (e.g., clavulanic acid), chloramphenicol, macrolides (e.g., erythromycin, azithromycin, clarithromycin), lincomycin, clindamycin, spectinomycin, polymyxin B, polymixins (e.g., polymyxin A, B, C, D, E1 (colistin A), or E2, colistin B or C) colistin, vancomycin, telavancin, bacitracin, isoniazid, rifampin, ethambutol, ethionamide, aminosalicylic acid, cycloserine, capreomycin, sulfones (e.g., dapsone, sulfoxone sodium, and the like), clofazimine, thalidomide.

In one embodiment, the compound of Formula (I) or (II), or pharmaceutically acceptable salt of Formula (I) or (II), is administered in combination with an aminoglycoside. In a further embodiment, the compound is a compound of Formula (I) or Formula (I) wherein R¹ is —(CH₂)₂—NH—(CH₂)₉—CH₃. The aminoglycoside, in a further embodiment, is dibekacin, K-4619, sisomicin, amikacin, dactimicin, isepamicin, rhodestreptomycin, apramycin, etimicin, KA-5685, sorbistin, arbekacin, framycetin, kanamycin, spectinomycin, astromicin, gentamicin, neomycin, sporaricin, bekanamycin, H107, netilmicin, streptomycin, boholmycin, hygromycin, paromomycin, tobramycin, brulamycin, hygromycin B, plazomicin, verdamicin, capreomycin, inosamycin, ribostamycin or vertilmicin. In a further embodiment, the aminoglycoside is amikacin or gentamicin. In a further embodiment, the aminoglycoside is gentamicin.

In another aspect, methods for treating bacterial infections, e.g., those caused by Gram-positive microorganisms, are provided. The method comprises, in one embodiment, administering to a patient in need of bacterial infection treatment, an effective amount of a compound of Formula (I) or (II), or a pharmaceutically acceptable salt of a compound of Formula (I) or (II). Administration in one embodiment, is intravenous or pulmonary.

The bacterial infection can comprise intracellular bacteria, planktonic bacteria and/or bacteria present in a biofilm.

Without wishing to be bound by a particular theory, it is believed that the R¹ groups conjugated to the glycopeptides provided herein facilitate cellular uptake of the glycopeptide at the site of infection, for example, macrophage uptake.

In one embodiment, the infection is a Gram-positive cocci infection, for example, a Staphylococcus, Enterococcus or Streptococcus infection. Streptococcus pnemoniae is treated, in one embodiment, in a patient that has been diagnosed with community-acquired pneumonia or purulent meningitis. An Enterococcus infection is treated, in one embodiment, in a patient that has been diagnosed with a urinary-catheter related infection. A Staphylococcus infection, e.g., S. aureus is treated in one embodiment, in a patient that has been diagnosed with mechanical ventilation-associated pneumonia.

Over the past few decades, there has been a decrease in the susceptibility of Gram-positive cocci to antibacterials for the treatment of infection. See, e.g., Alvarez-Lerma et al. (2006) Drugs 66, pp. 751-768, incorporated by reference herein in its entirety for all purposes. As such, in one aspect, the present invention addresses this need by providing a composition comprising an effective amount of a compound of Formula (I), Formula (II) or a pharmaceutically acceptable salt thereof, in a method for treating a patient in need thereof for a Gram-positive cocci infection that is resistant to a different antibacterial. For example, in one embodiment, the Gram-positive cocci infection is a penicillin resistant or a vancomycin resistant bacterial infection. In a further embodiment, the resistant bacterial infection is a methicillin-resistant Staphylococcus infection, e.g., methicillin-resistant S. aureus or a methicillin-resistant Staphylococcus epidermidis infection. In another embodiment, the resistant bacterial infection is an oxacillin-resistant Staphylococcus (e.g., S. aureus) infection, a vancomycin-resistant Enterococcus infection or a penicillin-resistant Streptococcus (e.g., S. pneumoniae) infection. In yet another embodiment, the Gram-positive cocci infection is a vancomycin-resistant enterococci (VRE), methicillin-resistant Staphylococcus aureus (MRSA), methicillin-resistant Staphylococcus epidermidis (MRSE), vancomycin resistant Enterococcus faecium also resistant to teicoplanin (VRE Fm Van A), vancomycin resistant Enterococcus faecium sensitive to teicoplanin (VRE Fm Van B), vancomycin resistant Enterococcus faecalis also resistant to teicoplanin (VRE Fs Van A), vancomycin resistant Enterococcus faecalis sensitive to teicoplanin (VRE Fs Van B), or penicillin-resistant Streptococcus pneumoniae (PSRP).

According to one embodiment, a method is provided to treat an infection due to a Gram-positive bacterium, including, but not limited to, genera Staphylococcus, Streptococcus, Enterococcus, Bacillus, Corynebaclerium, Nocardia, Clostridium, and Listeria. In one embodiment, the infection is due to a Gram-positive Cocci bacterium. In a further embodiment, the infection is a pulmonary infection. In another embodiment, the infection is a Clostridium difficile infection.

In even another embodiment, the bacterial infection is Propionibacterium Hi no. (skin acne), Eggerthella lenta (bacteremia) or Peptostreptococcus anaerobius (gynecological infection). In a further embodiment, the composition administered to the patient in need thereof comprises a compound of Formula (I) or Formula (II) wherein R¹ is —(CH₂)₂—NH—(CH₂)₉—CH₃ and X is O.

Staphylococcus is Gram positive non-motile bacteria that colonizes skin and mucus membranes. Staphylococci are spherical and occur in microscopic clusters resembling grapes. The natural habitat of Staphylococcus is nose; it can be isolated in 50% of normal individuals. 20% of people are skin carriers and 10% of people harbor Staphylococcus in their intestines. Examples of Staphylococci infections treatable with the methods and compositions provided herein, include S. aureus, S. epidermidis, S. auricularis, S. carnosus, S. haemolyticus, S. hyicus, S. intermedius, S. lugdunensis, S. saprophytics, S. sciuri, S. simulans, and S. warneri.

While there have been about 20 species of Staphylococcus reported, only Staphylococcus aureus and Staphylococcus epidermis are known to be significant in their interactions with humans.

In one embodiment, the Staphylococcus species is resistant to a penicillin such as methicillin. In a further embodiment, the Staphylococcus species is methicillin-resistant Staphylococcus aureus (MRSA) or methicillin-resistant Staphylococcus epidermidis (MRSE). The Staphylococcus infection, in another embodiment, is a methicillin-sensitive S. aureus (MSSA) infection, a vancomycin-intermediate S. aureus (VISA) infection, or a vancomycin-resistant S. aureus (VRSA) infection.

S. aureus colonizes mainly the nasal passages, but it may be found regularly in most anatomical locales, including skin oral cavity, and gastrointestinal tract. In one embodiment, a S. aureus infection is treated with one of the methods and/or compositions provided herein. In a further embodiment, the S. aureus infection is a methicillin-resistant Staphylococcus aureus (MRSA) infection. In another embodiment, the S. aureus infection is a S. aureus (VISA) infection, or a vancomycin-resistant S. aureus (VRSA) infection.

The S. aureus infection can be a healthcare associated, i.e., acquired in a hospital or other healthcare setting, or community-acquired.

In one embodiment, the Staphylococcal infection treated with one of the methods and/or compositions provided herein, causes endocarditis or septicemia (sepsis). As such, the patient in need of treatment with one of the methods and/or compositions provided herein, in one embodiment, is an endocarditis patient. In another embodiment, the patient is a septicemia (sepsis) patient.

In one embodiment, the bacterial infection is erythromycin-resistant (erm^{R}), vancomycin-intermediate S. aureus (VISA) heterogenous vancomycin-intermediate S. aureus (hVISA), S. epidermidis coagulase-negative staphylococci (CoNS), penicillin-intermediate S. pneumoniae (PISP), or penicillin-resistant S. pneumoniae (PRSP). In even a further embodiment, the administering comprises administering via inhalation. In even a further embodiment, the compound of Formula (I) or Formula (II) is a compound wherein R¹ is —(CH₂)₂—NH—(CH₂)₉—CH₃ or

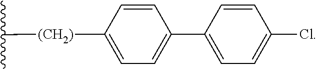

Streptococci are Gram-positive, non-motile cocci that divide in one plane, producing chains of cells. The primary pathogens include S. pyrogens and S. pneumoniae but other species can be opportunistic. S. pyrogens is the leading cause of bacterial pharyngitis and tonsillitis. It can also produce sinusitis, otitis, arthritis, and bone infections. Some strains prefer skin, producing either superficial (impetigo) or deep (cellulitis) infections.

S. pneumoniae is the major cause of bacterial pneumonia in adults, and in one embodiment, an infection due to S. pneumoniae is treated via one of the methods and/or compositions provided herein. Its virulence is dictated by its capsule. Toxins produced by streptococci include: streptolysins (S & O), NADase, hyaluronidase, streptokinase, DNAses, erythrogenic toxin (which causes scarlet fever rash by producing damage to blood vessels; requires that bacterial cells are lysogenized by phage that encodes toxin). Examples of Streptococcus infections treatable with the compositions and methods provided herein include, S. agalactiae, S. anginosus, S. bovis, S. canis, S. constellatus, S. dysgalactiae, S. equi, S. equinus, S. Mae, S. intermedins, S. mitis, S. mutans, S. oralis, S. parasanguinis, S. peroris, S. pneumoniae, S. pyogenes, S. ratti, S. salivarius, S. salivarius ssp. thermophilics, S. sanguinis, S. sobrinus, S. suis, S. uteris, S. vestibularis, S. viridans, and S. zooepidemicus.

The genus Enterococci consists of Gram-positive, facultatively anaerobic organisms that are ovoid in shape and appear on smear in short chains, in pairs, or as single cells. Enterococci are human pathogens that are increasingly resistant to antimicrobial agents. Examples of Enterococci treatable with the methods and compositions provided herein are E. avium, E. durans, E. faecalis, E. faecium, E. gallinarum, and E. solitarius.

In one embodiment of the methods provided herein, a patient in need thereof is treated for an Enterococcus faecalis (E. faecalis) infection. In a further embodiment, the infection is a pulmonary infection. In another embodiment, a patient in need thereof is treated for an Enterococcus faecium (E. faecium) infection. In a further embodiment, the infection is a pulmonary infection. In one embodiment, a patient in need thereof is treated for an Enterococcus infection that is resistant or sensitive to vancomycin or resistant or sensitive to penicillin. In a further embodiment, the infection is a E. faecalis or E. faecium infection.

Bacteria of the genus Bacillus are aerobic, endospore-forming, Gram-positive rods, and infections due to such bacteria are treatable via the methods and compositions provided herein. Bacillus species can be found in soil, air, and water where they are involved in a range of chemical transformations. In one embodiment, a method is provided herein to treat a Bacillus anthracis (B. anthracis) infection with a glycopeptide composition. Bacillus anthracis, the infection that causes Anthrax, is acquired via direct contact with infected herbivores or indirectly via their products. The clinical forms include cutaneous anthrax, from handling infected material, intestinal anthrax, from eating infected meat, and pulmonary anthrax from inhaling spore-laden dust. The route of administration of the glycopeptide will vary depending on how the patient acquires the B. anthracis infection. For example, in the case of pulmonary anthrax, the patient, in one embodiment, is treated via a dry powder inhaler (DPI), nebulizer or metered dose inhaler (MDI).

Several other Bacillus species, in particular, B. cereus, B. subtilis and B. licheniformis, are associated periodically with bacteremia/septicemia, endocarditis, meningitis, and infections of wounds, the ears, eyes, respiratory tract, urinary tract, and gastrointestinal tract, and are therefore treatable with the methods and compositions provided herein. Examples of pathogenic Bacillus species whose infection is treatable with the methods and compositions provided herein, include, but are not limited to, B. anthracis, B. cereus and B. coagulans.

Corynebacteria are small, generally non-motile, Gram-positive, non sporalating, pleomorphic bacilli and infections due to these bacteria are treatable via the methods provided herein. Corybacterium diphtheria is the etiological agent of diphtheria, an upper respiratory disease mainly affecting children, and is treatable via the methods provided herein. Examples of other Corynebacteria species treatable with the methods and compositions provided herein include Corynebacterium diphtheria, Corynebacterium pseudotuberculosis, Corynebacterium tenuis, Corynebacterium striatum, and Corynebacterium minutissimum.

The bacteria of the genus Nocardia are Gram-positive, partially acid-fast rods, which grow slowly in branching chains resembling fungal hyphae. Three species cause nearly all human infections: N. asteroides, N. brasiliensis, and N. caviae, and patients with such infections can be treated with the compositions and methods provided herein. Infection is by inhalation of airborne bacilli from an environmental source (soil or organic material). Other Nocardial species treatable with the methods provided herein include N. aerocolonigenes, N. africana, N. argentinensis, N. asteroides, N. blackwellu, N. brasiliensis, N. brevicalena, N. cornea, N. caviae, N. cerradoensis, N. corallina, N. cyriacigeorgica, N. dassonvillei, N. elegans, N. farcinica, N. nigiitansis, N. nova, N. opaca, N. otitidis-cavarium, N. paucivorans, N. pseudobrasiliensis, N. rubra, N. transvelencesis, N. uniformis, N vaccinii, and N. veterana.

Clostridia are spore-forming, Gram-positive anaerobes, and infections due to such bacteria are treatable via the methods and compositions provided herein. In one embodiment, one of the methods provided herein are used to treat a Clostridium tetani (C. tetani) infection, the etiological agent of tetanus. In another embodiment, one of the methods provided herein is used to treat a Clostridium botidinum (C. botidinum) infection, the etiological agent of botulism. In yet another embodiment, one of the methods provided herein is used to treat a C. perfringens infection, one of the etiological agents of gas gangrene. Other Clostridium species treatable with the methods of the present invention, include, C. difficile, C. perfringens, and/or C. sordellii. In one embodiment, the infection to be treated is a C. difficile infection.

Listeria are non spore-forming, nonbranching Gram-positive rods that occur individually or form short chains. Listeria monocytogenes (L. monocytogenes) is the causative agent of listeriosis, and in one embodiment, a patient infected with L. monocytogenes is treated with one of the methods and compositions provided herein. Examples of Listeria species treatable with the methods and compositions provided herein, include L. grayi, L. innocua, L. ivanovii, E. monocytogenes, E. seeligeri, L. murrayi, and L. welshimeri.

The bacterial infection in one embodiment, is a respiratory tract infection. In a further embodiment, the infection is a resistant bacterial infection, for example, one of the infections provided above. The patient treatable by the methods provided herein, in one embodiment, has been diagnosed with a community-acquired respiratory tract infection, e.g., pneumonia. In one embodiment, the bacterial infection treated in the pneumonia patient is a S. pneumoniae infection. In another embodiment, the bacterial infection treated in the pneumonia patient is Mycoplasma pneumoniae or a Legionella species. In another embodiment, the bacterial infection in the pneumonia patient is penicillin resistant, e.g., penicillin-resistant S. pneumoniae.

The bacterial infection, in one embodiment, is a hospital acquired infection (HAI), or acquired in another health care facility, e.g., a nursing home, rehabilitation facility, outpatient clinic, etc. Such infections are also referred to as nosocomial infections. In a further embodiment, the infection is a respiratory tract infection or a skin infection. In one embodiment, the HAI is pneumonia. In a further embodiment, the pneumonia is due to S. aureus, e.g., MRSA.

The inhalation delivery device employed in embodiments of the methods provided herein, e.g., methods for treating bacterial pulmonary infections, can be a nebulizer, dry powder inhaler (DPI), or a metered dose inhaler (MDI), or any other suitable inhalation delivery device known to one of ordinary skill in the art. The device can contain and be used to deliver a single dose of the composition or the device can contain and be used to deliver multi-doses of the composition of the present invention.

According to one embodiment, a dry powder particulate composition is delivered to a patient in need thereof via a metered dose inhaler (MDI), dry powder inhaler (DPI), atomizer, nebulizer or liquid dose instillation (LDI) technique to provide for glycopeptide delivery. With respect to inhalation therapies, those skilled in the art will appreciate that where a hollow and porous microparticle composition is employed, the composition is particularly amenable for delivery via a DPI. Conventional DPIs comprise powdered formulations and devices where a predetermined dose of medicament, either alone or in a blend with lactose carrier particles, is delivered as an aerosol of dry powder for inhalation.

The medicament is formulated in a way such that it readily disperses into discrete particles with an MMD between 0.5 to 20 μm, for example from 0.5-5 μm, and are further characterized by an aerosol particle size distribution less than about 10 μm mass median aerodynamic diameter (MMAD), and in some embodiments, less than 5.0 μm. The MMAD of the powders will characteristically range from about 0.5-10 μm, from about 0.5-5.0 μm, or from about 0.5-4.0 μm.

The powder is actuated either by inspiration or by some external delivery force, such as pressurized air. Examples of DPIs suitable for administration of the particulate compositions of the present invention are disclosed in U.S. Pat. Nos. 5,740,794, 5,785,049, 5,673,686, and 4,995,385 and PCT application Nos. 2000/72904, 2000/21594, and 2001/00263, the disclosure of each of which is incorporated by reference in their entireties for all purposes. DPI formulations are typically packaged in single dose units such as those disclosed in the aforementioned patents or they employ reservoir systems capable of metering multiple doses with manual transfer of the dose to the device.

The compositions disclosed herein may also be administered to the nasal or pulmonary air passages of a patient via aerosolization, such as with a metered dose inhaler (MDI). Breath activated MDIs are also compatible with the methods provided herein.

Along with the aforementioned embodiments, the compositions disclosed herein may be delivered to a patient in need thereof via a nebulizer, e.g., a nebulizer disclosed in PCT WO 99/16420, the disclosure of which is hereby incorporated in its entirety by reference, in order to provide an aerosolized medicament that may be administered to the pulmonary air passages of the patient. A nebulizer type inhalation delivery device can contain the compositions of the present invention as a solution, usually aqueous, or a suspension. For example, the prostacyclin compound or composition can be suspended in saline and loaded into the inhalation delivery device. In generating the nebulized spray of the compositions for inhalation, the nebulizer delivery device may be driven ultrasonically, by compressed air, by other gases, electronically or mechanically (e.g., vibrating mesh or aperture plate). Vibrating mesh nebulizers generate fine particle, low velocity aerosol, and nebulize therapeutic solutions and suspensions at a faster rate than conventional jet or ultrasonic nebulizers. Accordingly, the duration of treatment can be shortened with a vibrating mesh nebulizer, as compared to a jet or ultrasonic nebulizer. Vibrating mesh nebulizers amenable for use with the methods described herein include the Philips Respironics I-Neb®, the Omron MicroAir, the Nektar Aeroneb®, and the Pari eFlow®.

The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention, the nebulizer may be employed to ensure the sizing of particles is optimal for positioning of the particle within, for example, the pulmonary membrane.

Upon nebulization, the nebulized composition (also referred to as “aerosolized composition”) is in the form of aerosolized particles. The aerosolized composition can be characterized by the particle size of the aerosol, for example, by measuring the “mass median aerodynamic diameter” or “fine particle fraction” associated with the aerosolized composition. “Mass median aerodynamic diameter” or “MMAD” is normalized regarding the aerodynamic separation of aqua aerosol droplets and is determined by impactor measurements, e.g., the Andersen Cascade Impactor (ACI) or the Next Generation Impactor (NGI). The gas flow rate, in one embodiment, is 8 Liter per minute for the ACI and 15 liters per minute for the NGI.

“Geometric standard deviation” or “GSD” is a measure of the spread of an aerodynamic particle size distribution. Low GSDs characterize a narrow droplet size distribution (homogeneously sized droplets), which is advantageous for targeting aerosol to the respiratory system. The average droplet size of the nebulized composition provided herein, in one embodiment is less than 5 μm or about 1 μm to about 5 μm, and has a GSD in a range of 1.0 to 2.2, or about 1.0 to about 2.2, or 1.5 to 2.2, or about 1.5 to about 2.2.

“Fine particle fraction” or “FPF,” as used herein, refers to the fraction of the aerosol having a particle size less than 5 μm in diameter, as measured by cascade impaction. FPF is usually expressed as a percentage.

In one embodiment, the mass median aerodynamic diameter (MMAD) of the nebulized composition is about 1 μm to about 5 μm, or about 1 μm to about 4 μm, or about 1 μm to about 3 μm or about 1 μm to about 2 μm, as measured by the Andersen Cascade Impactor (ACI) or Next Generation Impactor (NGI). In another embodiment, the MMAD of the nebulized composition is about 5 μm or less, about 4 μm or less, about 3 μm or less, about 2 μm or less, or about 1 μm or less, as measured by cascade impaction, for example, by the ACI or NGI.

In one embodiment, the MMAD of the aerosol of the pharmaceutical composition is less than about 4.9 μm, less than about 4.5 μm, less than about 4.3 μm, less than about 4.2 μm, less than about 4.1 μm, less than about 4.0 μm or less than about 3.5 μm, as measured by cascade impaction.

In one embodiment, the MMAD of the aerosol of the pharmaceutical composition is about 1.0 μm to about 5.0 μm, about 2.0 μm to about 4.5 μm, about 2.5 μm to about 4.0 μm, about 3.0 μm to about 4.0 μm or about 3.5 μm to about 4.5 μm, as measured by cascade impaction (e.g., by the ACI or NGI).

In one embodiment, the FPF of the aerosolized composition is greater than or equal to about 50%, as measured by the ACI or NGI, greater than or equal to about 60%, as measured by the ACI or NGI or greater than or equal to about 70%, as measured by the ACI or NGI. In another embodiment, the FPF of the aerosolized composition is about 50% to about 80%, or about 50% to about 70% or about 50% to about 60%, as measured by the NGI or ACI.

In one embodiment, a metered dose inhalator (MDI) is employed as the inhalation delivery device for the compositions of the present invention. In a further embodiment, the prostacyclin compound is suspended in a propellant (e.g., hydroflourocarbon) prior to loading into the MDI. The basic structure of the MDI comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

In one embodiment, a dry powder inhaler (DPI) is employed as the inhalation delivery device for the compositions of the present invention.

In one embodiment, the DPI generates particles having an MMAD of from about 1 μm to about 10 μm, or about 1 μm to about 9 μm, or about 1 μm to about 8 μm, or about 1 μm to about 7 μm, or about 1 μm to about 6 μm, or about 1 μm to about 5 μm, or about 1 μm to about 4 μm, or about 1 μm to about 3 μm, or about 1 μm to about 2 μm in diameter, as measured by the NGI or ACI. In another embodiment, the DPI generates particles having an MMAD of from about 1 μm to about 10 μm, or about 2 μm to about 10 μm, or about 3 μm to about 10 μm, or about 4 μm to about 10 μm, or about 5 μm to about 10 μm, or about 6 μm to about 10 μm, or about 7 μm to about 10 μm, or about 8 μm to about 10 μm, or about 9 μm to about 10 μm, as measured by the NGI or ACI.

In one embodiment, the MMAD of the particles generated by the DPI is about 1 μm or less, about 9 μm or less, about 8 μm or less, about 7 μm or less, 6 μm or less, 5 μm or less, about 4 μm or less, about 3 μm or less, about 2 μm or less, or about 1 μm or less, as measured by the NGI or ACI.

In one embodiment, each administration comprises 1 to 5 doses (puffs) from a DPI, for example 1 dose (1 puff), 2 dose (2 puffs), 3 doses (3 puffs), 4 doses (4 puffs) or 5 doses (5 puffs). The DPI, in one embodiment, is small and transportable by the patient.

In one embodiment, the MMAD of the particles generated by the DPI is less than about 9.9 μm, less than about 9.5 μm, less than about 9.3 μm, less than about 9.2 μm, less than about 9.1 μm, less than about 9.0 μm, less than about 8.5 μm, less than about 8.3 μm, less than about 8.2 μm, less than about 8.1 μm, less than about 8.0 μm, less than about 7.5 μm, less than about 7.3 μm, less than about 7.2 μm, less than about 7.1 μm, less than about 7.0 μm, less than about 6.5 μm, less than about 6.3 μm, less than about 6.2 μm, less than about 6.1 μm, less than about 6.0 μm, less than about 5.5 μm, less than about 5.3 μm, less than about 5.2 μm, less than about 5.1 μm, less than about 5.0 μm, less than about 4.5 μm, less than about 4.3 μm, less than about 4.2 μm, less than about 4.1 μm, less than about 4.0 μm or less than about 3.5 μm, as measured by the NGI or ACI.

In one embodiment, the MMAD of the particles generated by the DPI is about 1.0 μm to about 10.0 μm, about 2.0 μm to about 9.5 μm, about 2.5 μm to about 9.0 μm, about 3.0 μm to about 9.0 μm, about 3.5 μm to about 8.5 μm or about 4.0 μm to about 8.0 μm.

In one embodiment, the FPF of the prostacyclin particulate composition generated by the DPI is greater than or equal to about 40%, as measured by the ACI or NGI, greater than or equal to about 50%, as measured by the ACI or NGI, greater than or equal to about 60%, as measured by the ACI or NGI, or greater than or equal to about 70%, as measured by the ACI or NGI. In another embodiment, the FPF of the aerosolized composition is about 40% to about 70%, or about 50% to about 70% or about 40% to about 60%, as measured by the NGI or ACI.

### EXAMPLES

The present invention is further illustrated by reference to the following Examples. However, it should be noted that these Examples, like the embodiments described above, are illustrative and are not to be construed as restricting the scope of the invention in any way.

### Example 1—Synthesis of Glycopeptide Derivative Via Reductive Amination

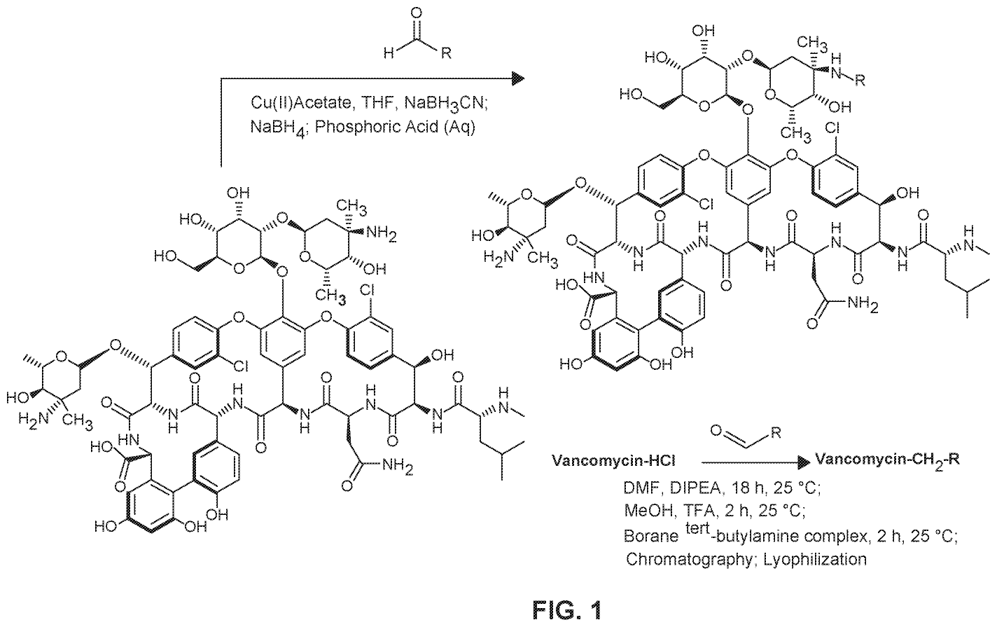

Glycopeptide derivatives were prepared as follows. The synthesis scheme is also provided at FIG. 1.

To a reactor vessel equipped with temperature control and agitation was added anhydrous DMF and DIPEA. The resulting solution was heated to 65° C. with agitation and Vancomycin HCl or telavancin HCl was added slowly in portions. Heating was continued until all of vancomycin HCl or telavancin HCl had dissolved (5-10 min).

The beige colored solution was allowed to cool after which a solution of the desired aldehyde dissolved in DMF was added over 5-10 min. The resulting solution was allowed to stir overnight, typically producing a clear red/yellow solution. MeOH and TFA were introduced and stirring was further continued for at least 2 h. At the end of the stirring period, the imine forming reaction mixture was analyzed by HPLC which was characteristically typical. Borane tert-butylamine complex was added in portions and the reaction mixture was stirred at ambient temperature for an additional 2 h after which an in-process HPLC analysis of the reaction mixture indicated a near quantitative reduction of the intermediate imine group. After the reaction was over, the reaction mixture was purified using reverse phase C18 column chromatography (Phenomenex Luna 10 uM PREP C18(2) 250×21.2 mm column) using gradients of water and acetonitrile, each containing 0.1% (v/v) of TFA. Fractions were evaluated using HPLC and then pertinent fractions containing the target product were pooled together for the isolation of the product via lyophilization. Typical products were isolated as fluffy white solids. The procedure is shown below in Scheme 1 with vancomycin HCl as a representative starting compound.

### Example 2—Synthesis of Vancomycin Derivative RV40 (Compound 40)

General synthesis: To a temperature controlled reactor vessel equipped with an overhead stirrer was added a suitable reaction solvent (DMF or DMA) and an organic base (typically DIPEA). The temperature was increased to approximately 60° C. and vancomycin HCl was added. The warm reaction mixture was agitated at elevated temperature for approximately 20 minutes at which point all vancomycin HCl had dissolved and the reaction mixture was returned to room temperature. To the reaction mixture was then added 9H-fluoren-9-ylmethyl N-decyl-N-(2-oxoethyl)carbamate (N-Fmoc-N-decylaminoacetaldehyde) dissolved in a suitable reaction solvent (DMF or DMA). The reaction mixture was agitated with an overhead stirrer overnight at which point a suitable reducing agent, acid catalyst (e.g., TFA), and a protic solvent (e.g., MeOH) were added. The reaction mixture was agitated by an overhead stirrer at room temperature for approximately two hours at which point solvent volume was reduced by half via rotary evaporation. To the concentrated reaction mixture was then added an organic base to remove the FMOC protecting group and yield crude product (Compound 40, also referred to as “RV40”). Solvent was then evaporated by rotary evaporation and the crude material was dry-packed using C18 silica and purified via reverse phase C18 flash chromatography to isolate product with >97% purity. Solvent was removed from the purified material using a combination of techniques including rotary evaporation, lyophilization, and spray drying to yield product (Compound 40 or RV40) as a white powder, typically in 40-75% overall yield. Suitable solvents include N,N-Dimethylacetamide, N,N-Dimethylformamide, N,N-Dimethylacetamide or a combination thereof. Suitable organic bases include N,N-diisopropylethylamine or trimethylamine. Suitable reducing agents include NaBH₄, NaBH₃CN, Borane-pyridine complex, or Borane-^{tert}butylamine complex. Suitable organic bases for FMOC deprotection include piperidine, methylamine, and ^{tert}butylamine.

Salt Forms: Control over the salt form and associated counter-ions for alkyl-vancomycin derivatives was managed by altering the acid species used during flash chromatography. Lactate, Acetate, HCl, and TFA salts have been prepared. To isolate free base derivatives of alkyl vancomycin derivatives the pH of purified material was adjusted between 7-8 to induce precipitation; purified free base material was then collected by filtration, rotary evaporation, lyophilization, or spray drying.

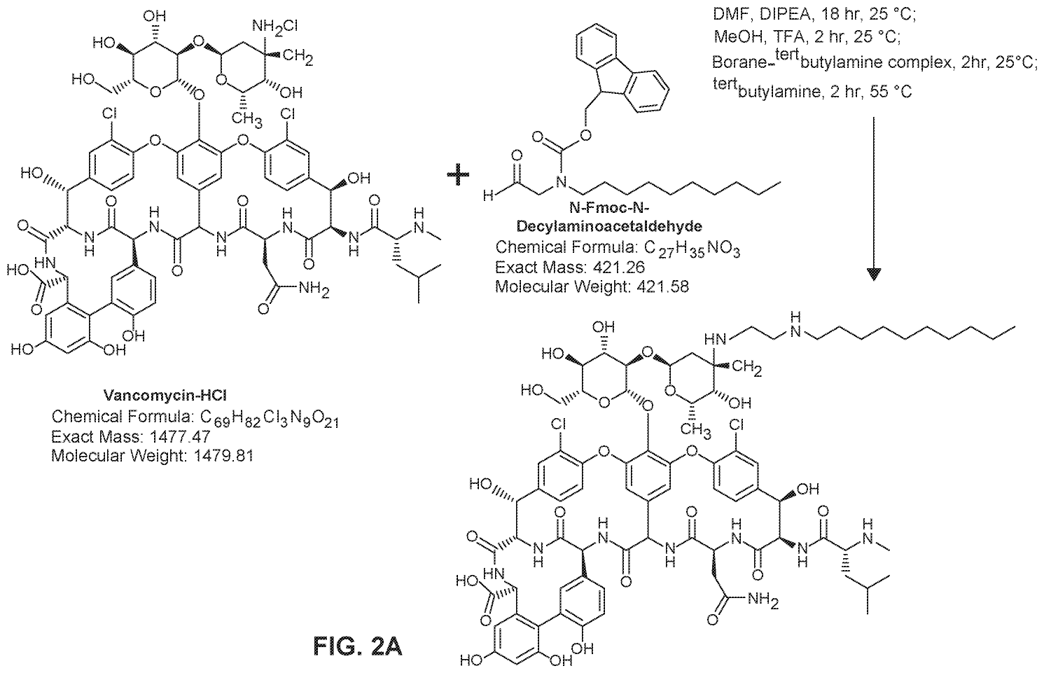

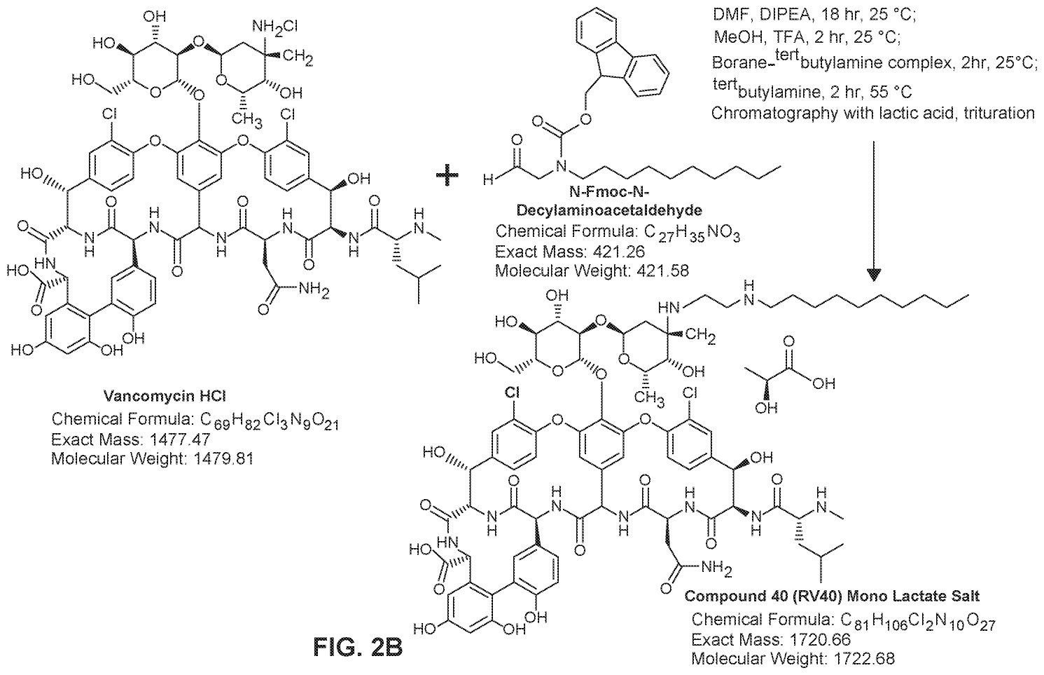

One synthetic scheme for arriving at compound 40 (RV40) is provided at FIG. 2 (top). Here, a jacketed 1 L reactor vessel was equipped with an overhead stirrer and connected to a recirculating water bath calibrated to 65° C. To the warm reaction vessel was added N,N-Dimethylformamide (75 mL) and DIPEA (640 μL, 3.7 mmol, 2.0 equivalents). Solvent was allowed to stir for 20 minutes and warmed to 65° C., at which point vancomycin HCl (2.70 g, 1.8 mmol, 1.00 equivalents) was added to the reaction mixture. Once all vancomycin HCl had dissolved the temperature was reduced to 25° C. and 9H-fluoren-9-ylmethyl N-decyl-N-(2-oxoethyl)carbamate (890 mg, 2.1 mmol, 1.15 equivalents) dissolved in N,N-Dimethylformamide (20 mL) was added. The reaction mixture was allowed to stir at 25° C. for 18 hr. To the reaction mixture was then added NaBH₃CN (330 mg, 5.3 mmol, 2.89 equivalents), MeOH (75 mL), and TFA (3.0 mL, 5.5 mmol, 3.00 equivalents). The reaction mixture was allowed to stir for 3 hr at RT at which point solvent volume was reduced by half via rotary evaporation. To the concentrated reaction mixture was then added piperidine (360 μL, 3.7 mmol, 2.00 equivalents) with stirring. Reaction progress was monitored by HPLC. Once HPLC analysis indicated complete deprotection, solvent was removed from the reaction mixture under reduced pressure to yield crude product (Compound 40) as an off-white solid. The crude material was dry-packed using C18 silica and purified via reverse phase C18 flash chromatography to isolate product with >97% purity.

### Example 3—Synthesis of Vancomycin Derivative RV40 (Compound 40)

General synthesis: To a temperature controlled reactor vessel equipped with an overhead stirrer was added a suitable reaction solvent (DMF or DMA) and an organic base (typically DIPEA). The temperature was increased to approximately 60° C. and vancomycin HCl was added. The warm reaction mixture was agitated at elevated temperature for approximately 20 minutes at which point all vancomycin HCl had dissolved and the reaction mixture was returned to room temperature. To the reaction mixture was then added 9H-fluoren-9-ylmethyl N-decyl-N-(2-oxoethyl)carbamate (N-Fmoc-N-decylaminoacetaldehyde) dissolved in a suitable reaction solvent (DMF or DMA). The reaction mixture was agitated with an overhead stirrer overnight. To the reaction mixture was added a protic solvent (e.g., MeOH) and an acid catalyst (e.g., TFA) and the reaction mixture was allowed to stir for 15 minutes prior to addition of a suitable reducing agent (e.g., borane tertbutylamine complex).

The reaction mixture was agitated by an overhead stirrer at room temperature for approximately two hours at which point an organic base (e.g., tertbutylamine) was added to remove the FMOC protecting group. The temperature was increased to 55° C. and the mixture was allowed to stir for 2 h. Solvent was then evaporated by rotary evaporation and the crude material was dry-packed using C18 silica and purified via reverse phase C18 flash chromatography to isolate product with >97% purity. Solvent was removed from the purified material using a combination of techniques including rotary evaporation, lyophilization, and spray drying to yield product (RV40) as a white powder, typically in 75% overall yield. Suitable solvents include N,N-Dimethylacetamide, N,N-Dimethylformamide, N,N-Dimethylacetamide or a combination thereof. Suitable organic bases include N,N-diisopropylethylamine or trimethylamine. Suitable reducing agents include NaBH₄, NaBH₃CN, Borane-pyridine complex, or Borane-^{tert}butylamine complex. Suitable organic bases for FMOC deprotection include piperidine, methylamine, and ^{tert}butylamine.

Salt Forms: Control over the salt form and associated counter-ions for alkyl-vancomycin derivatives was managed by altering the acid species used during flash chromatography. Lactate, Acetate, HCl, and TFA salts have been prepared. To isolate free base derivatives of the vancomycin derivative, the pH of purified material was adjusted between 7-8 to induce precipitation; purified free base material was then collected by filtration, rotary evaporation, lyophilization, or spray drying.

One synthetic scheme for arriving at compound 40 (RV40) is provided at FIG. 2, and is described in further detail below. To a 400 mL reactor vessel equipped with an overhead stirrer, a thermometer, and a pH meter was added DMF (50 mL) and DIPEA (1.17 mL, 6.73 mmol, 2.00 equivalents). The reaction mixture was heated to 55° C. at which point vancomycin HCl (5.0 g, 3.37 mmol, 1.0 equivalents) were added. The mixture was stirred at 55° C. for about 15 min., or until all of the vancomycin dissolved, at which point the temperature was reduced to 25° C. To the reaction mixture was added a solution of N-Fmoc-decylaminoacetaldehyde (1.63 g, 3.87 mmol, 1.15 equivalents) dissolved in DMF (16.32 mL). The reaction mixture was allowed to stir at 25° C. for 18 h. To the reaction mixture was added MeOH (14.0 mL) and TFA (1.03 mL, 13.46 mmol, 4.00 equivalent) and the mixture was allowed to stir at 25° C. for 15 min., at which point Borane tert-butylamine complex (294 mg, 3.37 mmol, 1.0 equivalents) were added. The reaction mixture was allowed to stir at 25° C. for 2 h, at which point tert-butylamine (4.24 mL, 40.38 mmol, 12.0 equivalents) was added, and the temperature was increased to 55° C. The reaction mixture was allowed to stir at 55° C. for 2 h. C18 functionalized silica gel was then added to the reaction mixture and solvent was removed under reduced pressure. The dry-packed material was purified using reverse phase C18 flash chromatography (Biotage® SNAP-KP-C18-HS column).

### Example 4—Preparation of Monolactate Salt of RV40

A 3 L three-necked flask was equipped with a mechanical stirrer, a nitrogen inlet, a condenser and an addition funnel. Anhydrous DMF (900 mL) and DIPEA (21.06 mL, 0.12 mol) were charged. The resulting solution was heated to 55-60° C. and vancomycin-HCl (90.0 g, 0.06 mol) was added in portions. Heating was continued until all of vancomycin-HCl had dissolved (15-30 min). The beige colored solution was allowed to cool to ambient temperature after which a solution of N-FMOC-N-decylaminoacetaldehyde (29.34 g, 0.069 mol) and DMF (293.4 mL) was added via the addition funnel over 5-10 min. The resulting solution was allowed to stir overnight to give a clear red-yellow solution. An in-process HPLC analysis of the reaction mixture at the end of the stirring period was typical. MeOH (252 mL) and TFA (18.54 mL, 0.24 mol) were introduced and stirring was further continued for at least 2 h. At the end of the stirring period, the inline forming reaction mixture was analyzed by HPLC which was characteristically typical. Borane tert-butylamine complex (5.28 g, 0.61 mol) was added in portions and the reaction mixture was stirred at ambient temperature for an additional 2 h after which an in-process HPLC analysis of the reaction mixture indicated a near quantitative reduction of the intermediate imine group with less than 3% of the unreacted vancomycin remaining. Tert-Butylamine (76.32 mL, 0.73 mol) was added via the addition funnel and the resulting reaction was heated to 55° C. The stirring was continued at 55° C. and progress of the FMOC group deprotection reaction was monitored by HPLC.

After the reaction was over (about 2 h), heating was removed and C18 silica gel (C-18 (Carbon 17%) 60A, 40-63 μm, 270 g) was added and the mixture was concentrated on a rotavap at 52° C./15 torr until free-flowing solids of C-18 silica adsorbed crude RV40 were obtained (3-7 h). The C-18 silica adsorbed crude RV40 (compound 40) was divided into three equal parts and each part-lot was purified by means of Biotage chromatography on a Biotage SNAP ULTRA C18 1850 g Cartridge (Biotage HP-Sphere C18 25 μm) using gradients of water and acetonitrile, each containing 0.1% (v/v) of an 85% L-(+)-Lactic acid solution in water, and collecting 240 mL fractions. Each part lot required ˜50 liters of eluents. After each Biotage run, the C-18 column was conditioned for the next run by running through 60 liters of methanol. Fractions were evaluated using HPLC and then pertinent fractions containing RV40were pooled together for the isolation of the product via lyophilization.

Lyophilization provided RV40 lactate salt as a white solid. The lyophilized RV40 lactate at this point typically contained excess lactic acid and also contained lactic acid related impurities arising from its self-condensation reactions. The isolated RV40 lactate from this run was combined with two other batches of similarly isolated lyophilized RV40 lactate to form a composite batch of RV40 lactate totaling 105 g (lot 637-140A). The excess lactic acid and its related impurities present in the above composite batch of RV40 lactate were removed via trituration with THF and then the final triturated material (RV40 mono lactate salts) was subjected to re-lyophilization to remove the trapped residual THF; both steps are described below.

A 5 L three-necked flask was equipped with a mechanical stirrer, a nitrogen inlet, and a condenser. RV40 lactate salts (105 g) and inhibitor-free anhydrous THF (1 L) were charged. The resulting mixture was stirred under nitrogen. After stirring overnight, the resulting mixture was filtered using a medium frit Buchner filter funnel. The filtered cake was washed with THF (250 mL). The filtered cake was dried on the filter funnel by pulling vacuum under nitrogen. After drying for 5 h the cake was analyzed by 1H NMR for the residual levels of lactic acid which were measured as 3.5 equivalents. The process of trituration with THF was repeated two more times after which the isolated product was determined to contain estimated 1 equivalent of lactic acid/lactate and THF. The isolated material was re-lyophilized to remove the residual THF as follows:

The above THF-triturated material was first dissolved in aqueous acetonitrile (3:1 water:acetonitrile) at a concentration of 8.1 mL per gram and then lyophilized in batches using multiple flasks. Typically, about 10-12 grams (maximum) of the material was charged into each 2 L flask followed by aqueous acetonitrile (125 mL) to prepare a solution which was lyophilized. At the end of the lyophilization and drying, product was analyzed by NMR for THF levels to determine whether lyophilization was needed to be repeated. In the current case, contents of each flask were lyophilized once more (after re-dissolving in 125 mL of aqueous acetonitrile) when no remaining THF could be detected by NMR. The final lyophilized product at this point contained an average of 0.8 wt. % acetonitrile as estimated by NMR. The contents of each flask were pulverized into smaller particles using spatula and then placed on high vacuum pumps to remove acetonitrile. No further reduction in acetonitrile levels was observed after 56-60 h on the vacuum pumps. Contents of each flasks were combined to provide a total of 74.3 g (35.5% yield based on the total conversion of 180 g of vancomycin-HCl) of a composite batch of RV40 mono lactate salts as white solid which was found to be >99 area % pure by HPLC and contained one equivalent of lactate as determined by 1HNMR (DMSO-d6) analysis. The water content in the product was found at 5.6 wt. % as determined by K-F analysis.

### Example 5—Synthesis of Vancomycin Derivative RV79

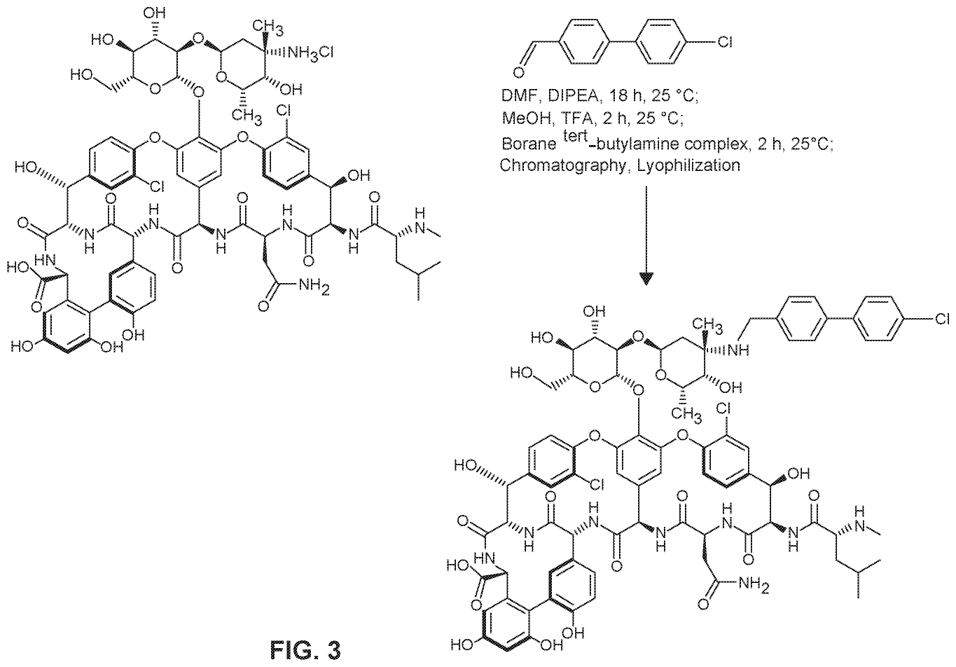

The synthesis scheme for arriving at the glycopeptide derivative RV79 is described below, and also provided at FIG. 3. To a 40 mL vial equipped a stir bar was added anhydrous DMF (20 mL) and DIPEA (0.20 mL). The resulting solution was heated to 65° C. on an incubated shaker and vancomycin-HCl (700 mg, 0.462 mmol) was added slowly in portions. Heating was continued until all of vancomycin-HCl had dissolved (5-10 min). The beige colored solution was allowed to cool to room temperature at which point 4′-Chloro-biphenyl-4-carbaldehyde (0.1 g, 0.462 mmol) was added to the reaction mixture. The reaction mixture was allowed to stir overnight. MeOH (1.5 mL) and TFA (0.14 mL, 1.8 mmol) were introduced and stirring was further continued for at least 2 h. Borane tert-butylamine complex (40 mg, 0.46 mmol) was added in portions and the reaction mixture was stirred at ambient temperature for an additional 2 h. After the reaction completed, the reaction mixture is purified using reverse phase C18 column chromatography (Phenomenex Luna 10 uM PREP C18(2) 250×21.2 mm column) using gradients of water and acetonitrile, each containing 0.1% (v/v) of TFA. Fractions were evaluated using HPLC and then pertinent fractions containing RV79 were pooled together for isolation of the product via lyophilization. The target compound, RV79 (81.2 mg, 0.05 mmol, 10% overall yield), was obtained as a white solid in >97% purity (by HPLC). The reaction scheme is shown at FIG. 3.

### Example 6—Synthesis of Alkyl-Vancomycin Derivatives

Alkyl vancomycin derivatives were prepared according to the procedure disclosed in Nagarajan et al., with slight modifications (Nagarajan et al. (1989). The Journal of Antibiotics 42(1), pp. 63-72, incorporated by reference herein in its entirety for all purposes).

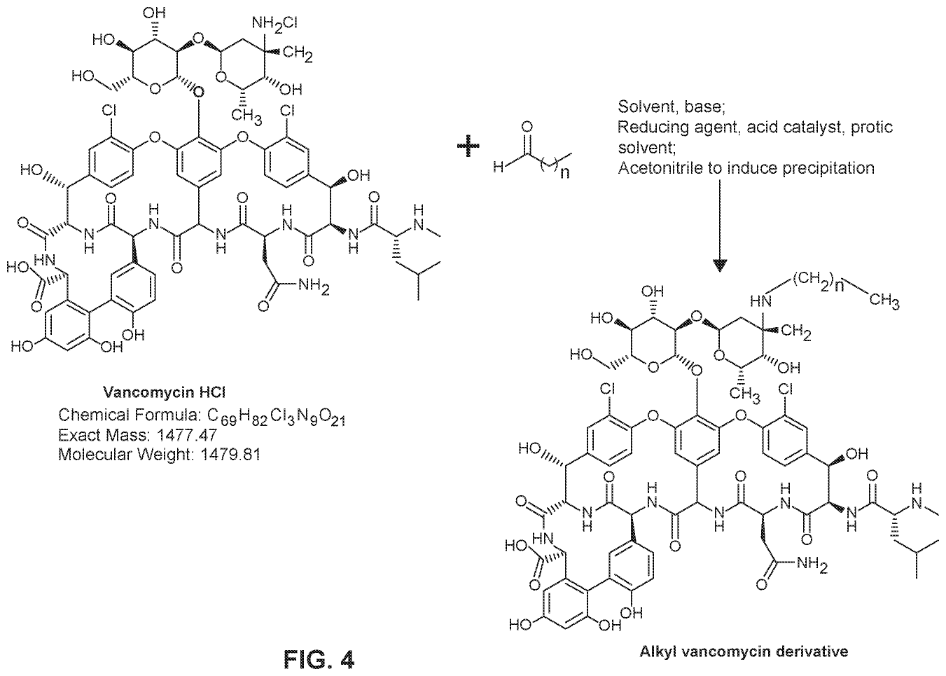

The general synthesis for alkyl vancomycin derivatives is shown in FIG. 4. Briefly, to a temperature-controlled reactor vessel was added vancomycin HCl, a suitable reaction solvent, an organic base, and the appropriate aldehyde. The reaction mixture was agitated with an overhead stirrer at elevated temperature and reaction progress was monitored via HPLC looking at consumption of vancomycin and imine formation. To the reactor vessel was then added a suitable reducing agent, acid catalyst (TFA), and a protic solvent (MeOH). The reaction mixture was agitated by an overhead stirrer for approximately 2 h. The reaction mixture was then either poured into water to induce precipitation of the alkyl vancomycin derivative, or solvent was removed under reduced pressure.

The crude material was dissolved in a suitable mobile phase and purified via preparative chromatography. Solvent was removed from the purified material using a combination of techniques including rotary evaporation, lyophilization, and spray drying to yield the vancomycin alkyl derivative as a white powder, typically in 40-60% overall yield. Suitable solvents include either N,N-Dimethylformamide or N,N-Dimethylacetamide. Suitable organic bases include N,N-diisopropylethylamine or trimethylamine. Suitable reducing agents include NaBH₄, NaBH₃CN, Borane-pyridine complex, or Borane-^{tert}butylamine complex.

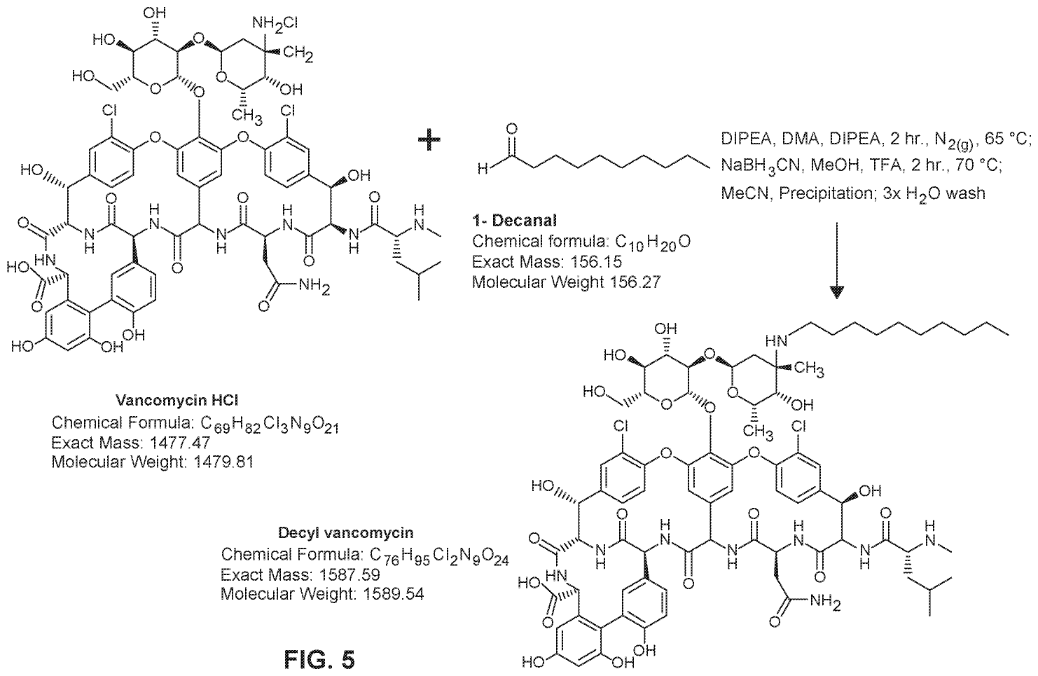

Synthesis of N-decyl Vancomycin (Compound 5): The synthetic route to Compound 5, decyl vancomycin, is provided at FIG. 5. A jacketed 1 L reactor vessel was equipped with an overhead stirrer and connected to a recirculating water bath calibrated to 65° C. To the warm reaction vessel was added N,N-Dimethylacetamide (160 mL) and DIPEA (6.8 mL, 39.0 mmol, 2.92 equivalents), the solvents were allowed to stir for approximately 20 minutes. Once the solvent temperature had reached 65° C., vancomycin HCl (19.8 g, 13.38 mmol, 1.00 equivalents) was added to the reactor vessel. To the reactor vessel was added 1-Decanal (2.54 mL, 13.50 mmol, 1.01 equivalents) and the reaction mixture was allowed to stir for 2 hours at 65° C. To the reaction mixture was then added NaBH₃CN (2.31 g, 36.77 mmol, 2.75 equivalents), MeOH (100 mL), and TFA (3.1 mL, 40.48 mmol, 3.03 equivalents). The reaction mixture was allowed to stir for 2 hours while cooling to room temperature. The reaction mixture was then poured into acetonitrile (1 L) to induce precipitation. The decant was removed and the remaining off-white slurry was centrifuged and decanted to remove excess solvent and produce a slurry containing N-decyl vancomycin and unreacted vancomycin. Crude N-decyl vancomycin as dissolved in 30:70 acetonitrile:H₂O with 0.05% HO Ac and purified using reverse phase C18 preparative HPLC. Pure fractions were subjected to rotary evaporation to remove organics and the flash-frozen and lyophilized to isolate purified N-decyl vancomycin as a fluffy white powder.

### Example 7—Synthesis of Chloroeremomycin Derivative RV79

To a 20 mL scintillation vial equipped with a stir bar was added chloroeremomycin and a solution of copper (II) acetate in MeOH. The reaction mixture was stirred at room temperature until the chloroeremomycin had dissolved. To the reaction mixture was then added the appropriate aldehyde and sodium cyanoborohydride as a 1M solution in THF. The reaction mixture was transferred to an incubated shaker set to 45° C. and reaction progress was monitored by HPLC. In some instances, it was necessary to add an additional aliquot of aldehyde reagent. The reaction mixture was allowed to shake overnight at 45° C. The reaction mixture was cooled to RT and sodium borohydride was added to convert residual aldehyde reagent to the corresponding alcohol. The pH was adjusted to between 7-8 using either acetic acid or 0.1M NaOH and volatile solvents were removed by blowing N₂ (g) with gentle heat. To the reaction mixture was added acetonitrile to precipitate the crude product as an off-white solid. The reaction mixture was centrifuged and the liquid was decanted. The solid was dissolved in 10% MeCN/H₂O containing 0.1% phosphoric acid to decomplex the copper at which point the solution briefly turned purple and then took on a yellow tinge. Preparatory HPLC was used to purify final product and LCMS was used to confirm compound identity and purity.

A diagram of the reaction is provided at FIG. 1, bottom.

### Example 8—C-Terminus Modification of Glycopeptide Derivative

To a round bottom flask equipped with a stir bar was added a glycopeptide derivative, a 1:1 solution of DMF:DMSO, and DIPEA. To the reaction mixture was then added HBTU and the appropriate amine (e.g., 3-(dimethylamino)-1-propylamine). Reaction progress was monitored by HPLC. Once complete, the reaction was quenched upon addition of 1:1 H₂O:MeOH. The crude material was then purified using reverse phase C18 preparatory HPLC. Purified fractions were lyophilized to yield the target products, typically as a white fluffy powder in modest yield and high purity.

### Example 9—Aminomethylation of Glycopeptide Resorcinol Group

To a reactor vessel equipped with overhead mechanical stirring and temperature control acetonitrile, water, and DIPEA are added. Stirring is initiated at room temperature and continues for about 10 minutes. The reaction mixture temperature is then reduced to −10° C., at which point an aqueous solution of 37% formaldehyde and the desired amine reagent is added to the reaction mixture. The reaction mixture is stirred at −10° C. for approximately 60 minutes, at which point the resorcinol containing glycopeptide is added as a solid. The reaction mixture is stirred overnight at 500 rpm while keeping the temperature constant at −10° C. Solvents are removed under reduced pressure to yield the crude material. The crude material is dissolved in a solution of 30% acetonitrile in water containing 0.1% TFA and is purified by preparative HPLC. Fractions collected from the preparative HPLC are assayed; pure fractions are combined and lyophilized to dryness to yield the target product as a white powder in high purity and modest yield.

### Example 10—Pharmacokinetics of Compounds of Formula (II) Administered Via Inhalation

Compounds subject to pharmacokinetic analysis are shown in Table 1, below.

TABLE 1

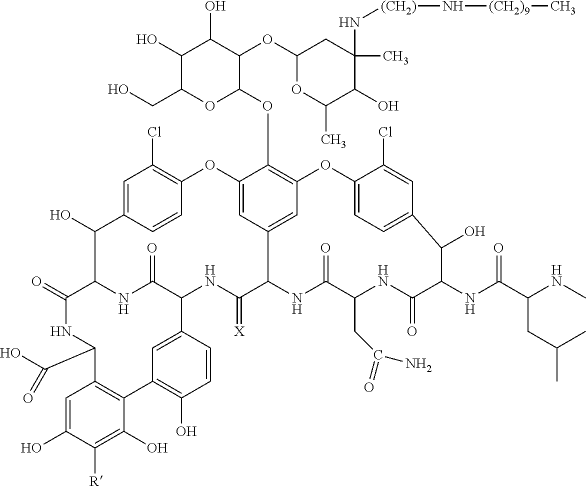

| R′ | Compound name |
|---|---|
| H | RV40 |
| CH₂—NH—CH₂—PO₃H₂ | Telavancin (TLV) |
| 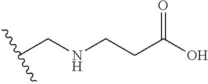 [SMILES: *CNCCC(=O)O] | RV104 |
| 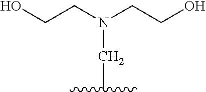 [SMILES: *CN(CCO)CCO] | RV106 |

120 h single dose in vivo PK experiments of nebulized inhaled compounds were performed in healthy male Sprague Dawley rats at target body-weight doses of 10 mg/kg (RV40) or 1.5 mg/kg (TLV, RV104, RV106), using a 12-port nose-only chamber (CH Technologies, Westwood, N.J., USA) equipped with an Aerogen Aeroneb Pro mesh nebulizer. The aerosol was provided to chamber at a flow rate of 6 L/min. Lungs were collected, and drug concentrations measured by HPLC-MS/MS.

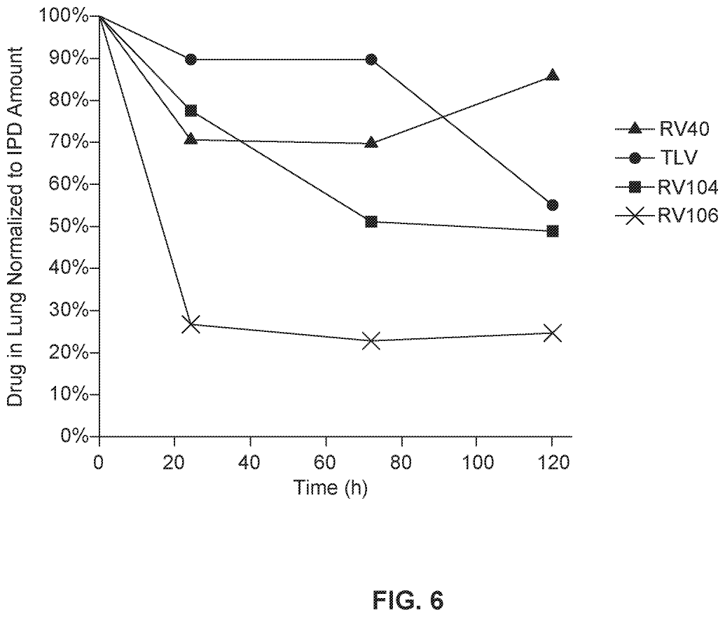

Although the semi-synthetic glycopeptide RV40 demonstrates potent antibacterial activity against gram positive pathogens including S. Aureus methicillin-susceptible and resistant isolates, when given by inhalation in a single dose in Sprague Dawley rats it has demonstrated a notably long half-life in lung tissue (t_{1/2}=300 h). Chemical modification of the compound to include an additional moiety at R=x was accomplished to improve reduce the compound's predictive hydrophobicity. Experimentally, this strategy has demonstrated a more favorable pharmacokinetic profile of the inhaled compound in terms of the relative lung tissue clearance of the modifications versus RV40 over the course of 120 h experiment as shown in FIG. 6.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A compound of Formula (I), or a pharmaceutically acceptable salt thereof: 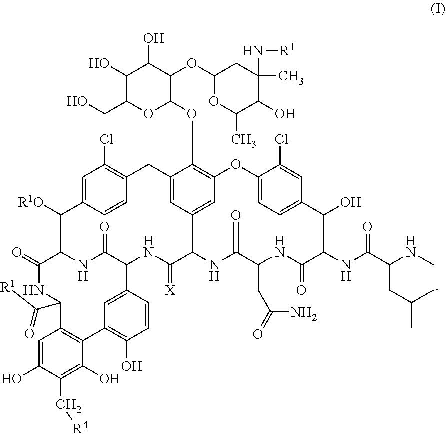
wherein,
R¹ is C₁-C₁₈ linear alkyl, C₁-C₁₈ branched alkyl, R⁵—Y—R⁶—(Z)ₙ, or 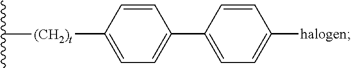
R² is —OH or —NH—(CH₂)_{q}—R⁷;
R³ is H or 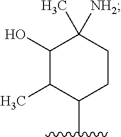 [SMILES: *C1CCC(C)(N)C(O)C1C]
R⁴ is diethanolamine, a monosaccharide, disaccharide, amino acid, or peptide, wherein the peptide has from 2 to 5 amino acids;
n is 1 or 2;
q is 1, 2, 3, 4, or 5;
t is 1, 2, 3, 4 or 5;
X is O, S, NH or H₂;
each Z is, independently, hydrogen, aryl, cycloalkyl, cycloalkenyl, heteroaryl, or heterocycl;
R⁵ and R⁶ are independently selected from the group consisting of alkylene, alkenylene and alkynylene, wherein the alkylene, alkenylene and alkynylene groups are optionally substituted with from 1 to 3 substituents selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl
R⁷ is —N(CH₂)₂; —N⁺(CH₂)₃; 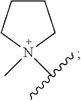
Y is oxygen, sulfur, —S—S—, —NR⁸—, —S(O)—, —SO₂—, —NR⁸C(O)—, —OSO₂—, —OC(O)—, —NR⁸SO₂—, —C(O)NR⁸—, —C(O)O—, —SO₂NR⁸—, —SO₂O—, —P(O)(OR⁸)O—, —P(O)(OR⁸)NR⁸—, —OP(O)(OR⁸)O—, —OP(O)(OR⁸)NR⁸—, —OC(O)O—, —NR⁸C(O)O—, —NR⁸C(O)NR⁸—, —OC(O)NR⁸— or —NR⁸SO₂NR⁸—; and
each R⁸ is independently selected from the group consisting of hydrogen, alkyl, substituted alkyl, alkenyl, substituted alkenyl, alkynyl, substituted alkynyl, cycloalkyl, substituted cycloalkyl, cycloalkenyl, substituted cycloalkenyl, aryl, heteroaryl and heterocyclic.

2. The compound of claim 1, or a pharmaceutically acceptable salt thereof, wherein R² is OH.

3-8. (canceled)

9. The compound of claim 1, or a pharmaceutically acceptable salt thereof, wherein R³ is H.

10-11. (canceled)

12. The compound of claim 1, or a pharmaceutically acceptable salt thereof, wherein X is O.

13-15. (canceled)

16. The compound of claim 1, or a pharmaceutically acceptable salt thereof, wherein R¹ is R⁵—Y—R⁶—(Z)ₙ.

17-18. (canceled)

19. The compound of claim 1, or a pharmaceutically acceptable salt thereof, wherein Y is —NH—.

20. The compound of claim 16, or a pharmaceutically acceptable salt thereof, wherein R⁶ is an unbranched C₄-C₁₆ alkylene, Z is H and n is 1.

21-22. (canceled)

23. The compound of claim 20, or a pharmaceutically acceptable salt thereof, wherein R⁶ is decylene.

24. The compound of claim 16, or a pharmaceutically acceptable salt thereof, wherein R¹ is (CH₂)₂—NH—(CH₂)₉—CH₃.

25-30. (canceled)

31. The compound of claim 16, or a pharmaceutically acceptable salt thereof, wherein R¹ is (CH₂)₂—Y—R⁶—(Z)ₙ, and (Z)ₙ is H.

32-55. (canceled)

56. The compound of claim 1, or a pharmaceutically acceptable salt thereof, wherein R⁴ is diethanolamine.

57. The compound of claim 1, or a pharmaceutically acceptable salt thereof, wherein R⁴ is an amino acid or a dipeptide.

58-60. (canceled)

61. The compound of claim 57, or a pharmaceutically acceptable salt thereof, wherein the amino acid is D-alanine.

62. The compound of claim 57, or a pharmaceutically acceptable salt thereof, wherein the amino acid is β-alanine, aspartic acid, glutamic acid, iminodiacetic acid, or glycine.

63-79. (canceled)

80. A method for treating a bacterial infection in a patient in need thereof, comprising administering to the patient an effective amount of a compound of claim 1, or a pharmaceutically acceptable salt thereof.

81. The method of claim 80, wherein the bacterial infection is a pulmonary bacterial infection.

82. The method of claim 81, wherein the administering comprises administering to the lungs of the patient via a nebulizer, a metered dose inhaler, or a dry powder inhaler.

83-96. (canceled)

97. The method of claim 80, wherein the bacterial infection is a Staphylococcus aureus (S. aureus) infection.

98. The method of claim 97, wherein the S. aureus infection is a methicillin-resistant S. aureus (MRSA) infection.

99-127. (canceled)

128. The method of claim 80, wherein the patient is a cystic fibrosis patient.

129-133. (canceled)
